(19) United States
(12) Patent Application Publication
(10) Pub. No.: US 2008/0255203 A1
(43) Pub. Date: Oct. 16, 2008

(54) HETEROCYCLIC COMPOUNDS AND THEIR METHODS OF USE

(75) Inventors: Chih-Hung Lee, Vernon Hills, IL (US); Jianguo Ji, Libertyville, IL (US); Tao Li, Grayslake, IL (US); Michael R. Schrimpf, Grayslake, IL (US); Kevin B. Sippy, Antioch, IL (US); Murali Gopalakrishnan, Libertyville, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 11/953,590

(22) Filed: Dec. 10, 2007

Related U.S. Application Data
(60) Provisional application No. 61/000,295, filed on Apr. 12, 2007.

(51) Int. Cl. A61K 31/4439; C07D 413/00; A61P 25/00; A61P 31/12

(52) U.S. Cl. 514/340; 546/269.1

(57) ABSTRACT

The invention relates to heterocyclic derivatives, compositions comprising such compounds, and methods of preventing or treating conditions and disorders using such compounds and compositions. The heterocyclic derivatives, more particularly can be substituted oxadiazole compounds and derivatives thereof.

### CROSS-REFERENCE TO RELATED APPLICATIONS

This application claims the benefit of U.S. Patent Application Ser. No. 61/000,295, filed Apr. 12, 2007, which is herein incorporated by reference in its entirety.

### BACKGROUND OF THE INVENTION

1. Technical Field

The invention relates to heterocyclic derivatives, compositions comprising such compounds, and methods of preventing or treating conditions and disorders using such compounds and compositions. The heterocyclic derivatives more particularly can be substituted oxadiazole compounds and derivatives thereof.

2. Description of Related Technology

The endogenous cholinergic neurotransmitter, acetylcholine, exert its biological effect via two types of cholinergic receptors, the muscarinic acetylcholine receptors (mAChR) and the nicotinic acetylcholine receptors (nAChR). Nicotinic acetylcholine receptors (nAChRs) are pentameric assemblies of subunits surrounding a central pore that gates the flux of Na⁺, K⁺ and Ca²⁺ ions. At least 12 subunit proteins, i.e. α2-α10 and β2-β4have been identified in neuronal tissues. These subunits provide for a great variety of homomeric and heteromeric combinations that account for the diverse receptor subtypes. For example, functional neuronal nAChR assemblies can be homomeric, comprising α7 or α8 or α9 subunits. Other subunits require heteromeric assembly, usually with at least one subunit (usually two or three) from the α group (α2, α3, α4, α6) and the remainder from the β group (β2, β4). In the central nervous system, α4β2-containing nAChR and α7-containing nAChR subtypes are the most widespread and mediate synaptic and, possibly, paracrine functions. These nAChRs are expressed at high levels in areas involved with learning and memory, and play key roles in modulating neurotransmission in these regions. Reduced cholinergic activity and dysregulation of nAChRs have been correlated with disease states involving cognitive deficits, progressive dementia, and epilepsy. Accordingly, these nAChRs are implicated in a range of physiological and patho-physiological functions related to cognitive function, learning and memory, reward, motor control, arousal and analgesia (reviewed in Gopalakrishnan, M and Briggs, C. A. Targets: Ion channels—Ligand-gated. Comprehensive Medicinal Chemistry II, Edited by David J. Triggle and John B. Taylor, Major Reference Works, Elsevier. Unit 2.22, pp 877-918, 2006).

Discovery of the important roles played by nAChRs in several CNS disorders has called attention to these membrane proteins and to ligands, or compounds, that are able to modulate, i.e. modify, the function of such membrane proteins. The prototypical nAChR agonist, nicotine, has itself been shown to improve attention and cognitive performance, reduce anxiety, normalize sensory gating, and effect neuroprotection. However, nicotine is not sufficiently selective among nAChRs and its utility is limited by side effects including seizures, irregular heartbeat, hypertension, and gastrointestinal effects. Accordingly, identification of compounds, agonists or allosteric modulators, that target distinct subtypes to retain the beneficial effects, while eliminating or decreasing adverse effects, continues to be an active area of research.

Neuronal nicotinic receptors, especially α4β2 neuronal nicotinic acetylcholine receptors (nAChRs) have been targeted for pain, cognitive disorders and various central nervous system diseases. Gene knockout, antisense and pharmacological studies have shown that α4 and β2 nAChRs are responsible for mediating nicotinic analgesia at supraspinal responses and spinal sites (Decker, M W, Rueter, L E and Bitner, R S (2005) Nicotinic acetylcholine receptor agonists: a potential new class of analgesics, Curr Top Med Chem., 4: 369-384). Ligands targeting α4β2 nAChRs have shown improvement in cognitive and attentive function in preclinical models and, more recently, in human disease states such as ADHD (Wilens, T. E., Verlinden, M. H., Adler, L. A., Wozniak, P. J. and West S. A., Biol Pscyhiatry, 59: 1065, 2006) and age-associated memory impairment (Dunbar, G C., Inglis, F., Kuchibatla, R., Sharma, T., Tomlinson, M. and Wamsley, J., J. Psyschopharmacol., 21: 171, 2007). A key goal in the discovery of novel nAChR compounds is to avoid ganglioinic α3* nAChRs, as the dose-limiting emetic liability of nonselective compounds may be attributed to activation of α3 containing nAChRs. α3* nAChRs in the dorsal motor nucleus of the vagus and in nucleus tractus solitarius have been implicated in gastric and blood pressure responses to nicotine injected locally (Ferreira M, Singh A, Dretchen K L, Kellar K J, and Gillis R A (2000) J. Pharmacol. Exp. Ther. 294:230-238).

Compounds with varying degrees of selectivity for α4β2 nAChRs over other nicotinic subtypes (α3, α7, α1-containing) have been discovered over the years for the treatment of pain and a range of psychiatric and neurological disorders especially involving cognitive deficits in attention, alertness and memory. These may include those conditions that may benefit from selective enhancement of cholinergic transmission such as attention deficit, psychotic disorders, selected pain syndromes, smoking cessation, substance abuse including alcohol, and those thought to involve reduced cholinergic function such as neurodegenerative disorders, central inflammatory or autoimmune disorders, brain trauma and cerbrovascular disease. Modulation of α4β2 nAChRs may be beneficial in an number of diseases including Alzheimer's disease, Mild Cognitive Impairment and related syndromes, Lewy Body dementia, vascular dementia, attention deficit/attention deficit-hyperactivity disorder, schizophrenia, bipolar and mood disorders, schizoaffective disorders, Tourrett's syndrome, brain trauma, vascular dementia, Parkinson's disease, Hungtinton's disease and conditions of substance abuse, including alcohol, and smoking cessation. Selected pain syndromes includes chronic pain that can be nociceptive, neuropathic, or both and originating from cancer, injury, surgery, or chronic conditions such as arthritis or nerve injury/disease. Neuropathic pain can be peripheral (painful peripheral mononeuropathy and polyneuropathy) or central (post stroke, following spinal cord injury) and can originate from nerve injury following a wide array of conditions or events, such as direct trauma to nerves, inflammation/neuritis/nerve compression, metabolic diseases (diabetes), infections (herpes zoster, HIV), tumors, toxins (chemotherapy), and primary neurological diseases.

Treatment with nAChR agonists, which act at the same site, as the endogenous transmitter ACh, may be problematic because ACh and other agonists not only activate, but also inhibits receptor activity through processes that include desensitization. Further, prolonged receptor activation may cause long-lasting inactivation. Thus, uncertainty exists whether chronic treatment with agonists in humans might provide suboptimal benefit due to sustained receptor activation and desensitization of the nAChRs. An alternate approach to target α4β2 nAChR function is by enhancing effects of the endogenous neurotransmitter acetylcholine via positive allosteric modulation. This approach provides an opportunity to (i) reinforce the endogenous cholinergic neurotransmission without directly activating the receptor like classical agonists, (ii) prevent receptor desensitization (iii) possibly resensitize inactivated receptors. Thus, the spatial and temporal characteristics of endogenous α4β2 receptor activation are preserved unlike agonists that that will tonically activate all receptors, leading to a non-physiological pattern of receptor activation.

In light of the evidence supporting the various therapeutic uses of nAChRs, it would be beneficial to discover novel allosteric modulators that could provide therapeutic benefits.

### SUMMARY OF THE INVENTION

The invention relates to heterocyclic compounds, compositions comprising such compounds, and method of using such compounds and compositions. In one aspect, the invention is directed to compounds of formula I

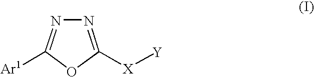

or a pharmaceutically acceptable salt or prodrug thereof, wherein

X is a bond, O, NR¹, S, or C₁-C₃ alkylene;

Y represents a monocyclic aryl, cycloalkyl, heterocycle, or heteroaryl group;

Ar¹ represents a monocyclic aryl or a heteroaryl group; and

R¹ is hydrogen, alkyl, haloalkyl or arylalkyl.

Another aspect of the invention relates to pharmaceutical compositions comprising compounds of the invention. Such compositions can be administered in accordance with a method of the invention, typically as part of a therapeutic regimen for treatment or prevention of conditions and disorders related to nAChR activity, and more particularly α4β2 nAChR positive allosteric modulator activity.

Yet another aspect of the invention relates to a method of modulating α4β2 nAChR positive allosteric modulator activity. The method is useful for treating, preventing or both treating and preventing conditions and disorders related to α4β2 nAChR positive allosteric modulator activity, particularly in mammals.

Such method is useful for treating, preventing or both treating and preventing conditions and disorders related to α4β2 nAChR activity in mammals. More particularly, the method is useful for conditions and disorders related to attention deficit disorder, attention deficit hyperactivity disorder (ADHD), Alzheimer's disease (AD), schizophrenia, mild cognitive impairment, age-associated memory impairment (AAMI), senile dementia, AIDS dementia, Pick's Disease, dementia associated with Lewy bodies, dementia associated with Down's syndrome, schizophrenia, amyotrophic lateral sclerosis, Huntington's disease, diminished CNS function associated with traumatic brain injury, acute pain, post-surgical pain, chronic pain, inflammatory pain, neuropathic pain, infertility, lack of circulation, need for new blood vessel growth associated with wound healing, more particularly circulation around a vascular occlusion, need for new blood vessel growth associated with vascularization of skin grafts, ischemia, inflammation, sepsis, wound healing, and other complications associated with diabetes, among other systemic and neuroimmunomodulatory activities. The method is useful for conditions and disorders related to conditions and disorders characterized by neuropsychological and cognitive dysfunction, for example in Alzheimer's disease, bipolar disorder, schizophrenia, schizoaffective disorder, and other related disorders characterized by neuropsychological and cognitive dysfunction, in particular. This method is also useful as treatment approaches for smoking cessation and substance abuse, including alcohol abuse.

Yet another aspect of the invention relates to a method for treating, preventing or both treating and preventing pain, particularly in mammals. The method is useful for treating nociceptive and neuropathic forms of pain, for example, chronic pain, analgesic pain, post-surgical pain, neuropathic pain, and diabetic neuropathy. Such compounds are particularly beneficial for reducing adverse ganglionic effects such as at the gastrointestinal systems (e.g. emesis) and enhance efficacy of nAChR ligands in such treatment.

A further aspect of the invention relates to a method of selectively modulating nAChR activity, for example α4β2 nAChR positive allosteric modulator activity, in combination with a nicotinic agonist or partial agonist to improve the tolerability of therapy using such nicotinic agonist or partial agonist. In such aspect, the invention relates to a composition comprising the α4β2 positive allosteric modulator and a neuronal nicotinic receptor ligand in admixture, or a method of administering an α4β2 positive allosteric modulator and a neuronal nicotinic receptor ligand in combination.

The compounds, compositions comprising the compounds, methods for using such compounds and composition, and further processes for preparing the compounds, as well as intermediates obtained in such processes, are further described herein.

### DETAILED DESCRIPTION OF THE INVENTION

### Definition of Terms

As used throughout this specification and the appended claims, the following terms have the following meanings:

The term “acyl hydrazide” as used herein, means a —C(O)NHNH₂ group.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkoxyalkyl” as used herein, means an alkoxyalkoxy group, as defined herein, appended to the parent molecular moiety through an alkyl group, as defined herein. Representative examples of alkoxyalkoxyalkyl include, but are not limited to, tert-butoxymethoxymethyl, ethoxymethoxymethyl, (2-methoxyethoxy)methyl, and 2-(2-methoxyethoxy)ethyl.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylcarbonyloxylalkyl” as used herein, means an alkylcarbonyloxy group, as defined herein, appended to the parent molecular moiety through an alkyl group.

The term “alkylene” means a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl” as used herein, means an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonylalkyl include, but are not limited to, methylsulfonylmethyl and ethylsulfonylmethyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkylthioalkyl” as used herein, means an alkylthio group, as defined herein, appended to the parent molecular moiety through an alkyl group, as defined herein. Representative examples of alkylthioalkyl include, but are not limited, methylthiomethyl and 2-(ethylthio)ethyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means phenyl, a bicyclic aryl or a tricyclic aryl. The bicyclic aryl is naphthyl, a phenyl fused to a cycloalkyl, or a phenyl fused to a cycloalkenyl. Representative examples of the bicyclic aryl include, but are not limited to, dihydroindenyl, indenyl, naphthyl, dihydronaphthalenyl, and tetrahydronaphthalenyl. The tricyclic aryl is anthracene or phenanthrene, or a bicyclic aryl fused to a cycloalkyl, or a bicyclic aryl fused to a cycloalkenyl, or a bicyclic aryl fused to a phenyl. Representative examples of tricyclic aryl ring include, but are not limited to, azulenyl, dihydroanthracenyl, fluorenyl, and tetrahydrophenanthrenyl.

The aryl groups of this invention can be substituted with 1, 2, 3, 4 or 5 substituents independently selected from alkenyl, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, alkylcarbonyloxy, alkylcarbonyloxyalkyl, alkylsulfinyl, alkylsulfinylalkyl, alkylsulfonyl, alkylsulfonylalkyl, alkylthio, alkylthioalkyl, alkynyl, arylalkyl, arylalkoxy, aryloxy, carboxy, carboxyalkyl, cyano, cyanoalkyl, formyl, formylalkyl, halogen, haloalkyl, haloalkoxy, hydroxy, hydroxyalkyl, mercapto, nitro, —NZ¹Z², and (NZ³Z⁴)carbonyl.

The term “arylalkoxy” as used herein, means an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “aryloxy” as used herein, means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “carbonyl” as used herein, means a —C(O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” as used herein, means a cyclic hydrocarbon containing from 3 to 8 carbons and containing at least one carbon-carbon double bond formed by the removal of two hydrogens. Representative examples of cycloalkenyl include, but are not limited to, 2-cyclohexen-1-yl, 3-cyclohexen-1-yl, 2,4-cyclohexadien-1-yl and 3-cyclopenten-1-yl.

The term “cycloalkyl” as used herein, means a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two adjacent or non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3.7}]nonane and tricyclo[3.3.1.1^{3.7}]decane (adamantane).

The cycloalkyl groups of the invention are optionally substituted with 1, 2, 3, 4 or 5 substituents selected from the group consisting of alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkylthioalkyl, alkynyl, carboxy, cyano, formyl, haloalkoxy, haloalkyl, halogen, hydroxy, hydroxyalkyl, mercapto, oxo, —NZ¹Z², and (NZ³Z⁴)carbonyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “formyl” as used herein, means a —C(O)H group.

The term “formylalkyl” as used herein, means a formyl group, as defined herein, appended to the parent molecular moiety through an alkyl group, as defined herein. Representative examples of formylalkyl include, but are not limited to, formylmethyl and 2-formylethyl.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic heteroaryl. The monocyclic heteroaryl is a 5 or 6 membered ring that contains at least one heteroatom selected from the group consisting of nitrogen, oxygen and sulfur. The 5 membered ring contains two double bonds and the 6 membered ring contains three double bonds. The 5 or 6 membered heteroaryl is connected to the parent molecular moiety through any carbon atom or any substitutable nitrogen atom contained within the heteroaryl, provided that proper valance is maintained. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any substitutable nitrogen atom contained within the bicyclic heteroaryl, provided that proper valance is maintained. Representative examples of bicyclic heteroaryl include, but are not limited to, azaindolyl, benzimidazolyl, benzofuranyl, benzoxadiazolyl, benzoisoxazole, benzoisothiazole, benzooxazole, 1,3-benzothiazolyl, benzothiophenyl, cinnolinyl, furopyridine, indolyl, indazolyl, isobenzofuran, isoindolyl, isoquinolinyl, naphthyridinyl, oxazolopyridine, quinolinyl, quinoxalinyl and thienopyridinyl,

The heteroaryl groups of the invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ¹Z² and (NZ³Z⁴)carbonyl. Heteroaryl groups of the invention that are substituted with a hydroxyl group may be present as tautomers. The heteroaryl groups of the invention encompasses all tautomers including non-aromatic tautomers.

The term “heterocycle” or “heterocyclic” as used herein, means a monocyclic heterocycle, a bicyclic heterocycle or a tricyclic heterocycle. The monocyclic heterocycle is a 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a 5 or 6 membered monocyclic heterocycle fused to a phenyl group, or a 5 or 6 membered monocyclic heterocycle fused to a cycloalkyl, or a 5 or 6 membered monocyclic heterocycle fused to a cycloalkenyl, or a 5 or 6 membered monocyclic heterocycle fused to a monocyclic heterocycle. The bicyclic heterocycle is connected to the parent molecular moiety through any carbon atom or any nitrogen atom contained within the bicyclic heterocycle. Representative examples of bicyclic heterocycle include, but are not limited to, 1,3-benzodioxolyl, 1,3-benzodithiolyl, 2,3-dihydro-1,4-benzodioxinyl, benzodioxolyl, 2,3-dihydro-1-benzofuranyl, 2,3-dihydro-1-benzothienyl, chromenyl and 1,2,3,4-tetrahydroquinolinyl. The tricyclic heterocycle is a bicyclic heterocycle fused to a phenyl, or a bicyclic heterocycle fused to a cycloalkyl, or a bicyclic heterocycle fused to a cycloalkenyl, or a bicyclic heterocycle fused to a monocyclic heterocycle. The tricyclic heterocycle is connected to the parent molecular moiety through any carbon atom or any nitrogen atom contained within the tricyclic heterocycle. Representative examples of tricyclic heterocycle include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The heterocycles of this invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, nitro, mercapto, oxo, —NZ¹Z² and (NZ³Z⁴)carbonyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “hydroxy-protecting group” or “O-protecting group” means a substituent which protects hydroxyl groups against undesirable reactions during synthetic procedures. Examples of hydroxy-protecting groups include, but are not limited to, substituted methyl ethers, for example, methoxymethyl, benzyloxymethyl, 2-methoxyethoxymethyl, 2-(trimethylsilyl)-ethoxymethyl, benzyl, and triphenylmethyl; tetrahydropyranyl ethers; substituted ethyl ethers, for example, 2,2,2-trichloroethyl and t-butyl; silyl ethers, for example, trimethylsilyl, t-butyldimethylsilyl and t-butyldiphenylsilyl; cyclic acetals and ketals, for example, methylene acetal, acetonide and benzylidene acetal; cyclic ortho esters, for example, methoxymethylene; cyclic carbonates; and cyclic boronates. Commonly used hydroxy-protecting groups are disclosed in T. W. Greene and P. G. M. Wuts, Protective Groups in Organic Synthesis, 3rd edition, John Wiley & Sons, New York (1999).

The term “lower alkenyl” as used herein, is a subset of alkenyl, as defined herein, and means an alkenyl group containing from 2 to 4 carbon atoms. Examples of lower alkenyl are ethenyl, propenyl, and butenyl.

The term “lower alkoxy” as used herein, is a subset of alkoxy, as defined herein, and means a lower alkyl group, as defined herein, appended to the parent molecular moiety through an oxygen atom, as defined herein. Representative examples of lower alkoxy include, but are not limited to, methoxy, ethoxy, propoxy, 2-propoxy, butoxy, and tert-butoxy.

The term “lower alkyl” as used herein, is a subset of alkyl as defined herein and means a straight or branched chain hydrocarbon group containing from 1 to 4 carbon atoms. Examples of lower alkyl are methyl, ethyl, n-propyl, iso-propyl, n-butyl, iso-butyl, sec-butyl, and tert-butyl.

The term “lower haloalkoxy” as used herein, is a subset of haloalkoxy, as defined herein, and means a straight or branched chain haloalkoxy group containing from 1 to 4 carbon atoms. Representative examples of lower haloalkoxy include, but are not limited to, trifluoromethoxy, trichloromethoxy, dichloromethoxy, fluoromethoxy, and pentafluoroethoxy.

The term “lower haloalkyl” as used herein, is a subset of haloalkyl, as defined herein, and means a straight or branched chain haloalkyl group containing from 1 to 4 carbon atoms. Representative examples of lower haloalkyl include, but are not limited to, trifluoromethyl, trichloromethyl, dichloromethyl, fluoromethyl, and pentafluoroethyl.

The term “methylenedioxy” as used herein, means a —OCH₂O— group wherein the oxygen atoms of the methylenedioxy are attached to the parent molecular moiety through two adjacent carbon atoms.

The term “nitrogen protecting group” as used herein, means those groups intended to protect an amino group against undesirable reactions during synthetic procedures. Preferred nitrogen protecting groups are acetyl, benzoyl, benzyl, benzyloxycarbonyl (Cbz), formyl, phenylsulfonyl, tert-butoxycarbonyl (Boc), tert-butylacetyl, trifluoroacetyl, and triphenylmethyl (trityl).

The term “mercapto” as used herein, means a —SH group.

The term “nitro” as used herein, means a —NO₂ group.

The term “NZ¹Z²” as used herein, means two groups, Z¹ and Z², which are appended to the parent molecular moiety through a nitrogen atom. Z¹ and Z² are each independently selected from the group consisting of hydrogen, alkyl, alkylcarbonyl, alkoxycarbonyl, aryl, arylalkyl, and formyl. In certain instances within the invention, Z¹ and Z² taken together with the nitrogen atom to which they are attached form a heterocyclic ring. Representative examples of NZ₁Z₂ include, but are not limited to, amino, methylamino, acetylamino, acetylmethylamino, phenylamino, benzylamino, azetidinyl, pyrrolidinyl and piperidinyl.

The term “NZ³Z⁴” as used herein, means two groups, Z³ and Z⁴, which are appended to the parent molecular moiety through a nitrogen atom. Z³ and Z⁴ are each independently selected from the group consisting of hydrogen, alkyl, aryl and arylalkyl. Representative examples of NZ³Z⁴ include, but are not limited to, amino, methylamino, phenylamino and benzylamino.

The term “oxo” as used herein, means a ═O moiety.

The term “sulfinyl” as used herein, means a —S(O)— group.

The term “sulfonyl” as used herein, means a —SO₂— group.

The term “tautomer” as used herein means a proton shift from one atom of a compound to another atom of the same compound wherein two or more structurally distinct compounds are in equilibrium with each other.

The term “positive allosteric modulator,” as used herein, means a compound that enhances activity of an endogenous, or naturally occurring, ligand, such as but not limited to Ach, or an exogenously administered agonist.

Although typically it may be recognized that an asterisk is used to indicate that the exact subunit composition of a receptor is uncertain, for example α4β2* indicates a receptor that contains the α4 and β2 subunits proteins in combination with other subunits.

### Compounds of the Invention

Compounds of the invention can have the formula (I)

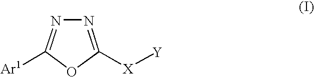

as described in the Summary of the Invention.

X is selected from a bond, O, NR¹, S, or C₁-C₃ alkylene, wherein R¹ is selected from hydrogen, alkyl, haloalkyl, and arylalkyl. Preferably, X is a bond. Preferably, R¹ is hydrogen or alkyl.

Y represents a monocyclic aryl, a cycloalkyl, a heterocycle, or a heteroaryl group, which can be substituted or unsubstituted with substituents. Examples of suitable heterocycle groups can include, but are not limited to, pyrrolidine, piperidine, and the like. Examples of suitable heteroaryl groups can include, but are not limited to, thienyl, furanyl, pyridinyl, pyrazinyl, and the like. A preferred monocyclic aryl group is substituted or unsubstituted phenyl. Suitable substituents for the monocyclic aryl, heterocycle, or heteroaryl group are, for example, alkyl, cycloalkyl, cycloalkylalkyl, halo, haloalkyl, hydroxyl, alkoxy, haloalkoxy, nitro, and cyano.

Ar₁ represents a monocyclic aryl, such as substituted or unsubstituted phenyl, or a heteroaryl group. Examples of suitable heteroaryl groups include, but are not limited, thienyl, furanyl, pyrrolyl, pyrazolyl, thiazolyl, 1,3,4-thiadiazolyl, and pyridinyl, each of which can be unsubstituted or substituted with one, two, or three substituents selected from alkyl, cycloalkyl, cycloalkylalkyl, halo, haloalkyl, hydroxyl, alkoxy, haloalkoxy, nitro, cyano, and amino.

In one embodiment, the compounds of the invention can have the formula (I) wherein X is a bond; Y is aryl, cycloalkyl, heterocycle, or heteroaryl; and Ar¹ is monocyclic aryl or a heteroaryl.

In another embodiment, the compounds of the invention can have the formula (I) wherein X is a bond; Y is monocyclic cycloalkyl, phenyl, thienyl, furyl, pyridinyl, pyrazinyl, pyrrolidinyl, or piperidinyl optionally substituted with one or more of the substituents selected from the group consisting of alkyl, halogen, haloalkyl, hydroxy, alkoxy, haloalkoxy, nitro and cyano; and Ar¹ is phenyl, thienyl, furyl, pyrrolyl, pyrazolyl, thiazolyl, 1,3,4-thiadiazolyl, pyrimidinyl, pyrazinyl, or pyridinyl optionally substituted with one or more of the substituents selected from the group consisting of alkyl, alkylcarbonyl, alkylsulfonyl, alkythio, alrylalkyl, aryloxy, arylalkyloxy, halogen, haloalkyl, hydroxy, alkoxy, haloalkoxy, nitro, cyano, and NZ¹Z², wherein Z¹ and Z² are as defined in the Definition of Terms.

In another embodiment, the compounds of the invention can have the formula (I) wherein X is a bond; Y is pyridyl; and Ar¹ is phenyl, pyrimidinyl, pyrazinyl, or pyridinyl optionally substituted with one or more of the substituents selected from the group consisting of alkyl, halogen, haloalkyl, hydroxy, alkoxy, haloalkoxy, nitro, cyano, and NZ¹Z², wherein Z¹ and Z² are as defined in the Definition of Terms.

Specific embodiments contemplated as part of the invention include, but are not limited to, compounds of formula (I), as defined, wherein the compound is:

2,5-di(pyridin-3-yl)-1,3,4-oxadiazole;

2-(5-bromopyridin-3-yl)-5-(pyridin-3-yl)-1,3,4-oxadiazole;

2-(pyridin-3-yl)-5-(4-(trifluoromethyl)phenyl)-1,3,4-oxadiazole;

2-(pyridin-3-yl)-5-o-tolyl-1,3,4-oxadiazole;

2-(pyridin-3-yl)-5-m-tolyl-1,3,4-oxadiazole;

2-(pyridin-3-yl)-5-p-tolyl-1,3,4-oxadiazole;

2-(5-(pyridin-3-yl)-1,3,4-oxadiazol-2-yl)phenol;

3-(5-(pyridin-3-yl)-1,3,4-oxadiazol-2-yl)phenol;

4-(5-(pyridin-3-yl)-1,3,4-oxadiazol-2-yl)phenol;

2-(3-methoxyphenyl)-5-(pyridin-3-yl)-1,3,4-oxadiazole;

2-(4-methoxyphenyl)-5-(pyridin-3-yl)-1,3,4-oxadiazole;

2-(2-fluorophenyl)-5-(pyridin-3-yl)-1,3,4-oxadiazole;

2-(3-fluorophenyl)-5-(pyridin-3-yl)-1,3,4-oxadiazole;

2-(4-fluorophenyl)-5-(pyridin-3-yl)-1,3,4-oxadiazole;

2-(2-chlorophenyl)-5-(pyridin-3-yl)-1,3,4-oxadiazole;

2-(3-chlorophenyl)-5-(pyridin-3-yl)-1,3,4-oxadiazole;

2-(4-chlorophenyl)-5-(pyridin-3-yl)-1,3,4-oxadiazole;

2-(2-bromophenyl)-5-(pyridin-3-yl)-1,3,4-oxadiazole;

2-(3-bromophenyl)-5-(pyridin-3-yl)-1,3,4-oxadiazole;

2-(4-bromophenyl)-5-(pyridin-3-yl)-1,3,4-oxadiazole;

3-(5-(pyridin-3-yl)-1,3,4-oxadiazol-2-yl)benzonitrile;

4-(5-(pyridin-3-yl)-1,3,4-oxadiazol-2-yl)benzonitrile;

N,N-dimethyl-3-(5-(pyridin-3-yl)-1,3,4-oxadiazol-2-yl)aniline;

N,N-dimethyl-4-(5-(pyridin-3-yl)-1,3,4-oxadiazol-2-yl)aniline;

2-(pyridin-3-yl)-5-(3-(trifluoromethyl)phenyl)-1,3,4-oxadiazole;

2-(pyridin-3-yl)-5-(3-(trifluoromethoxy)phenyl)-1,3,4-oxadiazole;

2-(4-phenoxyphenyl)-5-(pyridin-3-yl)-1,3,4-oxadiazole;

2-(4-(benzyloxy)phenyl)-5-(pyridin-3-yl)-1,3,4-oxadiazole;

2-(3,4-dimethylphenyl)-5-(pyridin-3-yl)-1,3,4-oxadiazole;

2-(3,5-dimethylphenyl)-5-(pyridin-3-yl)-1,3,4-oxadiazole;

2-(2,5-dimethylphenyl)-5-(pyridin-3-yl)-1,3,4-oxadiazole;

2-(2,4-dimethylphenyl)-5-(pyridin-3-yl)-1,3,4-oxadiazole;

2-(3,4-dimethylphenyl)-5-(pyridin-3-yl)-1,3,4-oxadiazole;

2-(2,3-dimethoxyphenyl)-5-(pyridin-3-yl)-1,3,4-oxadiazole;

2-(2,4-dimethoxyphenyl)-5-(pyridin-3-yl)-1,3,4-oxadiazole;

2-(2,5-dimethoxyphenyl)-5-(pyridin-3-yl)-1,3,4-oxadiazole;

2-(2,4-dimethoxyphenyl)-5-(pyridin-3-yl)-1,3,4-oxadiazole;

2-(3,5-dimethoxyphenyl)-5-(pyridin-3-yl)-1,3,4-oxadiazole;

2-(benzo[d][1,3]dioxol-5-yl)-5-(pyridin-3-yl)-1,3,4-oxadiazole;

2-(pyridin-3-yl)-5-(3,4,5-trimethoxyphenyl)-1,3,4-oxadiazole;

2-(3,4-dichlorophenyl)-5-(pyridin-3-yl)-1,3,4-oxadiazole;

2-(2,4-dichlorophenyl)-5-(pyridin-3-yl)-1,3,4-oxadiazole;

2-(2,5-dichlorophenyl)-5-(pyridin-3-yl)-1,3,4-oxadiazole;

2-(3,4-dichlorophenyl)-5-(pyridin-3-yl)-1,3,4-oxadiazole;

5-methyl-2-(5-(pyridin-3-yl)-1,3,4-oxadiazol-2-yl)phenol;

2-methyl-5-(5-(pyridin-3-yl)-1,3,4-oxadiazol-2-yl)phenol;

2-(3-fluoro-2-methylphenyl)-5-(pyridin-3-yl)-1,3,4-oxadiazole;

2-(5-fluoro-2-methylphenyl)-5-(pyridin-3-yl)-1,3,4-oxadiazole;

2-(3-fluoro-4-methylphenyl)-5-(pyridin-3-yl)-1,3,4-oxadiazole;

2-(2,3-difluorophenyl)-5-(pyridin-3-yl)-1,3,4-oxadiazole;

2-(2,4-difluorophenyl)-5-(pyridin-3-yl)-1,3,4-oxadiazole;

2-(2,5-difluorophenyl)-5-(pyridin-3-yl)-1,3,4-oxadiazole;

2-(3,5-difluorophenyl)-5-(pyridin-3-yl)-1,3,4-oxadiazole;

1-(4-(5-(pyridin-3-yl)-1,3,4-oxadiazol-2-yl)phenyl)ethanone;

2-(4-isopropylphenyl)-5-(pyridin-3-yl)-1,3,4-oxadiazole;

2-(3-methoxy-4-methylphenyl)-5-(pyridin-3-yl)-1,3,4-oxadiazole;

2-(4-ethoxyphenyl)-5-(pyridin-3-yl)-1,3,4-oxadiazole;

2-(4-(methylthio)phenyl)-5-(pyridin-3-yl)-1,3,4-oxadiazole;

2-(3-fluoro-4-methoxyphenyl)-5-(pyridin-3-yl)-1,3,4-oxadiazole;

2-(naphthalen-1-yl)-5-(pyridin-3-yl)-1,3,4-oxadiazole;

2-(naphthalen-2-yl)-5-(pyridin-3-yl)-1,3,4-oxadiazole;

4-chloro-2-(5-(pyridin-3-yl)-1,3,4-oxadiazol-2-yl)phenol;

2-(4-tert-butylphenyl)-5-(pyridin-3-yl)-1,3,4-oxadiazole;

N-(4-(5-(pyridin-3-yl)-1,3,4-oxadiazol-2-yl)phenyl)acetamide;

2-(4-propoxyphenyl)-5-(pyridin-3-yl)-1,3,4-oxadiazole;

2-(4-isopropoxyphenyl)-5-(pyridin-3-yl)-1,3,4-oxadiazole;

2-(5-chloro-2-methoxyphenyl)-5-(pyridin-3-yl)-1,3,4-oxadiazole;

2-(4-fluoronaphthalen-1-yl)-5-(pyridin-3-yl)-1,3,4-oxadiazole;

N,N-diethyl-4-(5-(pyridin-3-yl)-1,3,4-oxadiazol-2-yl)aniline;

2-(4-butoxyphenyl)-5-(pyridin-3-yl)-1,3,4-oxadiazole;

2-(2-methoxy-4-(methylthio)phenyl)-5-(pyridin-3-yl)-1,3,4-oxadiazole;

2-(4-(methylsulfonyl)phenyl)-5-(pyridin-3-yl)-1,3,4-oxadiazole;

2-(2-chloro-5-(methylthio)phenyl)-5-(pyridin-3-yl)-1,3,4-oxadiazole;

2-(2-fluoro-5-(trifluoromethyl)phenyl)-5-(pyridin-3-yl)-1,3,4-oxadiazole;

2-(2-chloro-5-(trifluoromethyl)phenyl)-5-(pyridin-3-yl)-1,3,4-oxadiazole;

2-(2-phenethylphenyl)-5-(pyridin-3-yl)-1,3,4-oxadiazole;

2-(2-bromo-5-methoxyphenyl)-5-(pyridin-3-yl)-1,3,4-oxadiazole;

2-(5-bromo-2-chlorophenyl)-5-(pyridin-3-yl)-1,3,4-oxadiazole;

2-(2-iodophenyl)-5-(pyridin-3-yl)-1,3,4-oxadiazole;

2-(3-iodophenyl)-5-(pyridin-3-yl)-1,3,4-oxadiazole;

2-(4-iodophenyl)-5-(pyridin-3-yl)-1,3,4-oxadiazole;

2-(pyridin-3-yl)-5-(pyrimidin-5-yl)-1,3,4-oxadiazole;

2-(5-methylpyrazin-2-yl)-5-(pyridin-3-yl)-1,3,4-oxadiazole;

2-(2-chloro-6-methylpyridin-3-yl)-5-(pyridin-3-yl)-1,3,4-oxadiazole;

2-(2-methyl-6-(trifluoromethyl)pyridin-3-yl)-5-(pyridin-3-yl)-1,3,4-oxadiazole;

2-(2-(ethylthio)pyridin-3-yl)-5-(pyridin-3-yl)-1,3,4-oxadiazole;

2-(2,6-dimethoxypyridin-3-yl)-5-(pyridin-3-yl)-1,3,4-oxadiazole;

2-(2-(methylthio)pyridin-3-yl)-5-(pyridin-3-yl)-1,3,4-oxadiazole;

5-chloro-3-(5-(pyridin-3-yl)-1,3,4-oxadiazol-2-yl)pyridin-2-ol;

2-(2,6-dichloro-5-fluoropyridin-3-yl)-5-(pyridin-3-yl)-1,3,4-oxadiazole;

2-(2,5-dichloropyridin-3-yl)-5-(pyridin-3-yl)-1,3,4-oxadiazole;

2-(6-chloropyridin-3-yl)-5-(pyridin-3-yl)-1,3,4-oxadiazole;

2-(2,6-dichloropyridin-3-yl)-5-(pyridin-3-yl)-1,3,4-oxadiazole;

2-(2-chloropyridin-3-yl)-5-(pyridin-3-yl)-1,3,4-oxadiazole; and

2-(pyridin-3-yl)-5-(quinolin-3-yl)-1,3,4-oxadiazole.

Compound names are assigned by using Struct=Name naming algorithm, which is part of the CHEMDRAW® ULTRA v. 9.0.7 software suite.

### Methods of Preparing Compounds of the Invention

The compounds and processes of the invention will be better understood in connection with the following synthetic schemes and examples, which illustrate a means by which the compounds of the present invention can be prepared. All citations identified with respect to the preparation of the compounds are incorporated herein by reference.

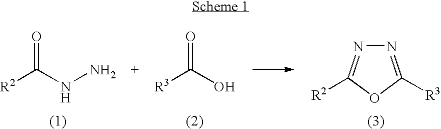

As outlined in Scheme 1, compounds of formula (1) can be reacted with compounds of formula (2) in POCl₃ at temperatures from 40-100° C. over 1-24 hours to provide compounds of formula (3); wherein R² is Ar¹ and R³ is Y, or R² is Y and R³ is Ar¹. Alternatively, compounds of formula (1) can be reacted with compounds of formula (2) in the presence of triphenylphosphine, which may optionally be polymer bound, and trichloroacetonitrile in acetonitrile. The mixture may be heated in a microwave oven at 100-175° C. for 5-30 minutes as described by Wang, Y.; Sauer, D. R.; Djuric, S. W. Tetrahedron. Lett. 2006, 47, 105-108. Another alternative includes combining compounds of formula (1) and compounds of formula (2) in a solvent such as methylene chloride in the presence of 2-chloro-1,3-dimethylimidazolinium chloride and a base such as triethylamine at 15-35° C. for 10-120 hours as described by Isobe, T.; Ishikawa, T. J. Org. Chem. 1999, 64, 6989-6992.

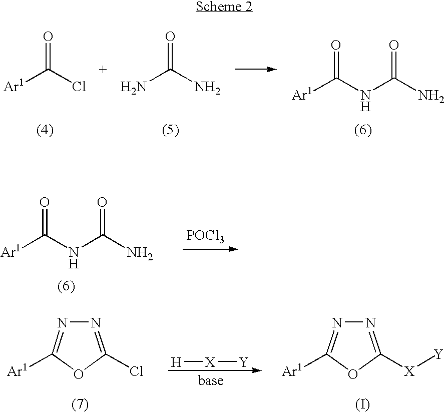

As outlined in Scheme 2, compounds of formula (4) can be reacted with urea, (5), in a solvent such as dichloromethane in the presence of a base such as triethylamine at 25-40° C. for 1-12 hours to provide compounds of formula (6) as described in Sobol, E., Bialer, M.; Yagen, B. J. Med. Chem. 2004, 47, 4316-4326. Alternatively, compounds of formula (4) and (5) may be combined in pyridine at 20-110° C. for 1-24 hours to provide compounds of formula (6). Compounds of formula (6) can be treated with POCl₃ at 25-100° C. for 1-24 hours to provide compounds of formula (7). Compounds of formula (7) can be reacted with H—X—Y in the presence of a base such as lithium bis(trimethylsilyl)amide, sodium bis(trimethylsilyl)amide, potassium bis(trimethylsilyl)amide, potassium t-butoxide, sodium hydride, potassium carbonate, sodium carbonate, cesium or carbonate in a solvent such as tetrahydrofuran, 1-methyl-2-pyrrolidinone, dimethyl sulfoxide, or acetonitrile at temperatures from −20° C. to 150° C. over 1-48 hours to provide compounds of formula (I).

The compounds and intermediates of the invention may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

The compounds and processes of the present invention will be better understood in connection with the following Examples, which are intended as an illustration of and not a limitation upon the scope of the invention.

### EXAMPLES

### Synthesis of 2,5-disubstituted-1,3,4-oxadiazoles

Method A: A carboxylic acid (0.5 mmol) and an acyl hydrazide (0.5 mmol) were combined in POCl₃ (2 mL) and stirred at 80-90° C. for 2-4 hours. The reaction mixture was then cooled down to ambient temperature and poured into ice water (10-20 g) and basified with saturated aqueous sodium carbonate to pH=8-9. The resultant precipitate was filtered, dried and purified with chromatography on silica gel to provide the corresponding 2,5-disubstituted-1,3,4-oxadiazole. The free base was then dissolved in EtOAc (5-10 mL) and treated with HCl (Aldrich, 4 M in dioxane, 2-3 eq.) at ambient temperature for 5-10 hours. The precipitate was filtered and dried to provide the corresponding 2,5-disubstituted-1,3,4-oxadiazole hydrochloric acid salt.

Method B: A Smith Process vial (0.5-2 ml) was charged with a stir bar. To the vessel were added a carboxylic acid (0.1 mmol), nicotinic hydrazide (Aldrich, 13.7 mg, 0.1 mmol), PS—PPh₃ (Fluka, 2.2 mmol/g, 136 mg, 0.3 mmol) and MeCN (anhydrous, Aldrich, 2 mL), followed by CCl₃CN (Aldrich, 28.8 mg, 0.20 mmol). The reaction vessel was sealed and heated to 150° C. for 15 minutes using an Emrys™ Optimizer Microwave (Personal Chemistry, www.personalchemistry.com). After cooling, the reaction vessel was uncapped and the resin was removed by filtration. The mixture was purified by preparative HPLC [Waters, column: Nova-Pak® HR C18 6 μm 60 Å Prep-Pak® (25 mm×100 mm), solvent: MeCN/water (v.1% TFA), 5/95 to 95/5, flow rate of 40 mL/min. Fractions were collected based upon UV signal threshold, and selected fractions were subsequently analyzed by flow injection analysis mass spectrometry using positive APCl ionization on a Finnigan LCQ using 70:30 MeOH:10 mM NH₄OH(aq) at a flow rate of 0.8 mL/min.]. Some mixtures were purified by an alternative preparative HPLC method [Waters, column: Sunfire OBD C8 5 μm (30 mm×75 mm); solvent: MeCN/10 mM aqueous ammonium acetate, 10/90 to 100/0; flow rate of 50 mL/min. Fractions were collected based upon target mass signal threshold, and selected fractions were subsequently analyzed by flow injection analysis mass spectrometry using the previously described method.].

### Example 1

### 2,5-di(pyridin-3-yl)-1,3,4-oxadiazole bishydrochloride

Prepared according to Method A. ¹H NMR (300 MHz, MeOH-d₄) δ 8.35 (dd, J=8.1, 5.8 Hz, 2 H), 9.13 (d, J=5.6 Hz, 2 H), 9.36 (d, J=7.9 Hz, 2 H), 9.75 (s, 2 H) ppm; MS (DCl/NH₃) m/z 225 (M+H)⁺.

### Example 2

### 2-(5-bromopyridin-3-yl)-5-(pyridin-3-yl)-1,3,4-oxadiazole bishydrochloride

Prepared according to Method A. ¹H NMR (300 MHz, MeOH-d₄) δ 8.29 (dd, J=8.1, 5.8 Hz, 1 H), 8.77-8.87 (m, 1 H), 8.96 (d, J=2.4 Hz, 1 H), 9.08 (dd, J=5.8, 1.4 Hz, 1 H), 9.28 (dt, J=8.1, 2.0, 1.8 Hz, 1 H), 9.35 (d, J=2.0 Hz, 1 H), 9.67 (d, J=2.0 Hz, 1 H) ppm; MS (DCl/NH₃) m/z 305 (M+H)⁺, 303 (M+H)⁺.

### Example 3

### 2-(pyridin-3-yl)-5-(4-(trifluoromethyl)phenyl)-1,3,4-oxadiazole trifluoroacetate

Prepared according to Method B. ¹H NMR (500 MHz, DMSO-d₆) δ 7.71 (dd, J=8.2, 4.9 Hz, 1 H), 8.03 (d, J=8.2 Hz, 2 H), 8.40 (d, J=8.2 Hz, 2 H), 8.55 (dt, J=8.1, 2.0, 1.8 Hz, 1 H), 8.85 (d, J=3.7 Hz, 1 H), 9.33 (d, J=1.2 Hz, 1 H) ppm, MS (DCl/NH₃) m/z 292 (M+H)⁺.

### Example 4

### 2-(pyridin-3-yl)-5-o-tolyl-1,3,4-oxadiazole trifluoroacetate

Prepared according to Method B. ¹H NMR (500 MHz, DMSO-d₆) δ 2.71 (s, 3 H), 7.44-7.53 (m, 2 H), 7.53-7.59 (m, 1 H), 7.71 (dd, J=8.1, 4.7 Hz, 1 H), 8.11 (dd, J=7.9, 1.2 Hz, 1 H), 8.53 (dt, J=8.1, 2.0, 1.8 Hz, 1 H), 8.84 (d, J=4.3 Hz, 1 H), 9.30 (s, 1 H) ppm; MS (DCl/NH₃) m/z 238 (M+H)⁺.

### Example 5

### 2-(pyridin-3-yl)-5-m-tolyl-1,3,4-oxadiazole trifluoroacetate

Prepared according to Method B. ¹H NMR (500 MHz, DMSO-d₆) δ 2.44 (s, 3 H), 7.47-7.51 (m, 1 H), 7.54 (t, J=7.6 Hz, 1 H), 7.70 (dd, J=8.2, 4.9 Hz, 1 H), 7.93-8.04 (m, 2 H), 8.53 (dt, J=7.9, 2.0 Hz, 1 H), 8.83 (d, J=3.7 Hz, 1 H), 9.31 (s, 1 H) ppm; MS (DCl/NH₃) m/z 238 (M+H)⁺.

### Example 6

### 2-(pyridin-3-yl)-5-p-tolyl-1,3,4-oxadiazole trifluoroacetate

Prepared according to Method B. ¹H NMR (500 MHz, DMSO-d₆) δ 2.43 (s, 3 H), 7.47 (d, J=7.9 Hz, 2 H), 7.72 (dd, J=7.6, 4.6 Hz, 1 H), 8.06 (d, J=8.2 Hz, 2 H), 8.54 (dt, J=8.2, 1.9 Hz, 1 H), 8.83 (d, J=3.4 Hz, 1 H), 9.31 (d, J=1.5 Hz, 1 H) ppm; MS (DCl/NH₃) m/z 238 (M+H)⁺.

### Example 7

### 2-(5-(pyridin-3-yl)-1,3,4-oxadiazol-2-yl)phenol trifluoroacetate

Prepared according to Method B. ¹H NMR (500 MHz, DMSO-d₆) δ 7.09 (t, J=7.5 Hz, 1 H), 7.14 (d, J=8.5 Hz, 1 H), 7.49-7.57 (m, 1 H), 7.72 (dd, J=7.9, 4.9 Hz, 1 H), 7.98 (dd, J=7.8, 1.7 Hz, 1 H), 8.51 (dt, J=7.9, 1.8 Hz, 1 H), 8.84 (d, J=4.0 Hz, 1 H), 9.29 (s, 1 H) ppm; MS (DCl/NH₃) m/z 240 (M+H)⁺.

### Example 8

### 3-(5-(pyridin-3-yl)-1,3,4-oxadiazol-2-yl)phenol trifluoroacetate

Prepared according to Method B. ¹H NMR (500 MHz, DMSO-d₆) δ 7.08 (dd, J=8.2, 1.8 Hz, 1 H), 7.47 (t, J=7.9 Hz, 1 H), 7.52-7.56 (m, 1 H), 7.62 (d, J=7.9 Hz, 1 H), 7.71 (dd, J=7.9, 4.9 Hz, 1 H), 8.53 (dt, J=7.9, 1.8 Hz, 1 H), 8.83 (d, J=4.0 Hz, 1 H), 9.30 (s, 1 H) ppm; MS (DCl/NH₃) m/z 240 (M+H)⁺.

### Example 9

### 4-(5-(pyridin-3-yl)-1,3,4-oxadiazol-2-yl)phenol trifluoroacetate

Prepared according to Method B. ¹H NMR (500 MHz, DMSO-d₆) δ 7.02 (d, J=9.2, Hz, 1 H), 7.72 (dd, J=7.9, 4.9 Hz, 1 H), 8.02 (d, J=8.8 Hz, 2 H), 8.53 (dt, J=8.1, 1.8 Hz, 1 H), 8.83 (d, J=4.3 Hz, 1 H), 9.30 (s, 1 H) ppm; MS (DCl/NH₃) m/z 240 (M+H)⁺.

### Example 10

### 2-(3-methoxyphenyl)-5-(pyridin-3-yl)-1,3,4-oxadiazole trifluoroacetate

Prepared according to Method B. ¹H NMR (500 MHz, DMSO-d₆) δ 3.89 (s, 3 H), 7.25 (dd, J=8.1, 2.3 Hz, 1 H), 7.58 (t, J=7.9 Hz, 1 H), 7.66-7.69 (m, 1 H), 7.72 (dd, J=7.8, 5.0 Hz, 1 H), 7.76 (d, J=7.6 Hz, 1 H), 8.56 (dt, J=8.1, 1.9 Hz, 1 H), 8.84 (d, J=4.3 Hz, 1 H), 9.34 (s, 1 H) ppm; MS (DCl/NH₃) m/z 254 (M+H)⁺.

### Example 11

### 2-(4-methoxyphenyl)-5-(pyridin-3-yl)-1,3,4-oxadiazole trifluoroacetate

Prepared according to Method B. ¹H NMR (500 MHz, DMSO-d₆) δ 3.88 (s, 3 H), 7.20 (dt, J=9.5, 2.6, Hz, 1 H). 7.69 (dd, J=8.1, 4.7 Hz, 1 H), 8.12 (dt, J=9.5, 2.6 Hz, 1 H), 8.50 (dt, J=8.3, 1.8 Hz, 1 H), 8.82 (d, J=4.0 Hz, 1 H), 9.29 (s, 1 H) ppm; MS (DCl/NH₃) m/z 254 (M+H)⁺.

### Example 12

### 2-(2-fluorophenyl)-5-(pyridin-3-yl)-1,3,4-oxadiazole trifluoroacetate

Prepared according to Method B. ¹H NMR (500 MHz, DMSO-d₆) δ 7.46-7.58 (m, 2 H), 7.71-7.79 (m, 2 H), 8.21 (td, J=7.6, 1.7 Hz, 1 H), 8.55 (dt, J=8.1, 1.7 Hz, 1 H), 8.86 (d, J=4.3 Hz, 1 H), 9.31 (s, 1 H) ppm; MS (DCl/NH₃) m/z 242 (M+H)⁺.

### Example 13

### 2-(3-fluorophenyl)-5-(pyridin-3-yl)-1,3,4-oxadiazole trifluoroacetate

Prepared according to Method B. ¹H NMR (500 MHz, DMSO-d₆) δ 7.53 (td, J=8.5, 2.4 Hz, 1 H), 7.67-7.77 (m, 2 H), 7.99 (dt, J=9.5, 2.0 Hz, 1 H), 8.04 (d, J=7.9 Hz, 1 H), 8.57 (dt, J=7.9, 1.8 Hz, 1 H), 8.85 (d, J=2.4 Hz, 1 H), 9.35 (s, 1 H) ppm; MS (DCl/NH₃) m/z 242 (M+H)⁺.

### Example 14

### 2-(4-fluorophenyl)-5-(pyridin-3-yl)-1,3,4-oxadiazole trifluoroacetate

Prepared according to Method B. ¹H NMR (500 MHz, DMSO-d₆) δ 7.46-7.55 (m, 2 H), 7.70 (dd, J=7.6, 4.6 Hz, 1 H), 8.21-8.28 (m, 2 H), 8.53 (dt, J=8.0, 1.9 Hz, 1 H), 8.83 (d, J=3.7 Hz, 1 H), 9.31 (s, 1 H) ppm; MS (DCl/NH₃) m/z 242 (M+H)⁺.

### Example 15

### 2-(2-chlorophenyl)-5-(pyridin-3-yl)-1,3,4-oxadiazole trifluoroacetate

Prepared according to Method B. ¹H NMR (500 MHz, DMSO-d₆) δ 7.63 (td, J=7.6, 1.2 Hz, 1 H), 7.68-7.75 (m, 2 H), 7.77 (dd, J=8.2, 1.2 Hz, 1 H), 8.17 (dd, J=7.8, 1.7 Hz, 1 H), 8.52 (dt, J=8.0, 1.9 Hz, 1 H), 8.85 (d, J=4.0 Hz, 1 H), 9.29 (s, 1 H) ppm; MS (DCl/NH₃) m/z 260 (M+H)⁺, 258 (M+H)⁺.

### Example 16

### 2-(3-chlorophenyl)-5-(pyridin-3-yl)-1,3,4-oxadiazole trifluoroacetate

Prepared according to Method B. ¹H NMR (500 MHz, DMSO-d₆) δ 7.67-7.72 (m, 2 H), 7.75 (ddd, J=8.1, 2.1, 1.1 Hz, 1 H), 8.15 (d, J=7.6 Hz, 1 H), 8.22 (t, J=1.8 Hz, 1 H), 8.55 (dt, J=8.0, 1.9 Hz, 1 H), 8.84 (d, J=4.0 Hz, 1 H), 9.34 (s, 1 H) ppm; MS (DCl/NH₃) m/z 260 (M+H)⁺, 258 (M+H)⁺.

### Example 17

### 2-(4-chlorophenyl)-5-(pyridin-3-yl)-1,3,4-oxadiazole trifluoroacetate

Prepared according to Method B. ¹H NMR (500 MHz, DMSO-d₆) δ 7.69-7.75 (m, 3 H), 8.20 (dt, J=8.8, 2.3 Hz, 2 H), 8.54 (dt, J=8.2, 1.9 Hz, 1 H), 8.84 (d, J=4.3 Hz, 1 H), 9.32 (s, 1 H) ppm; MS (DCl/NH₃) m/z 260 (M+H)⁺, 258 (M+H)⁺.

### Example 18

### 2-(2-bromophenyl)-5-(pyridin-3-yl)-1,3,4-oxadiazole trifluoroacetate

Prepared according to Method B. ¹H NMR (500 MHz, DMSO-d₆) δ 7.61 (td, J=7.8, 1.8 Hz, 1 H), 7.67 (td, J=7.5, 1.2 Hz, 1 H), 7.72 (dd, J=7.6, 5.2 Hz, 1 H), 7.94 (dd, J=7.9, 1.2 Hz, 1 H), 8.11 (dd, J=7.8, 1.7 Hz, 1 H), 8.51 (dt, J=8.0, 1.9 Hz, 1 H), 8.85 (d, J=3.4 Hz, 1 H), 9.28 (d, J=1.5 Hz, 1 H) ppm; MS (DCl/NH₃) m/z 304 (M+H)⁺, 302 (M+H)⁺.

### Example 19

### 2-(3-bromophenyl)-5-(pyridin-3-yl)-1,3,4-oxadiazole trifluoroacetate

Prepared according to Method B. ¹H NMR (500 MHz, DMSO-d₆) δ 7.63 (t, J=7.9 Hz, 1 H), 7.70 (dd, J=7.9, 4.9 Hz, 1 H), 7.89 (ddd, J=7.9, 1.8, 0.9 Hz, 1 H), 8.19 (d, J=7.9 Hz, 1 H), 8.35 (t, J=1.8 Hz, 1 H), 8.56 (dt, J=8.2, 1.9 Hz, 1 H), 8.84 (d, J=3.7 Hz, 1 H), 9.34 (d, J=1.2 Hz, 1 H) ppm; MS (DCl/NH₃) m/z 304 (M+H)⁺, 302 (M+H)⁺.

### Example 20

### 2-(4-bromophenyl)-5-(pyridin-3-yl)-1,3,4-oxadiazole trifluoroacetate

Prepared according to Method B. ¹H NMR (500 MHz, DMSO-d₆) δ 7.70 (dd, J=8.1, 4.7 Hz, 1 H), 7.87 (dt, J=8.8, 2.3 Hz, 1 H), 8.12 (dt, J=8.8, 2.3 Hz, 1 H), 8.53 (dt, J=7.9, 1.8 Hz, 1 H), 8.84 (d, J=4.0 Hz, 1 H), 9.32 (s, 1 H) ppm; MS (DCl/NH₃) m/z 304 (M+H)⁺, 302 (M+H)⁺.

### Example 21

### 3-(5-(pyridin-3-yl)-1,3,4-oxadiazol-2-yl)benzonitrile trifluoroacetate

Prepared according to Method B. ¹H NMR (500 MHz, DMSO-d₆) δ 7.72 (dd, J=7.9, 4.9 Hz, 1 H), 7.87 (t, J=7.9 Hz, 1 H), 8.13 (d, J=7.9 Hz, 1 H), 8.49 (d, J=8.2 Hz, 1 H), 8.59 (dt, J=7.9, 1.8 Hz, 1 H), 8.64 (s, 1 H), 8.85 (d, J=3.4 Hz, 1 H), 9.37 (d, J=1.5 Hz, 1 H) ppm; MS (DCl/NH₃) m/z 249 (M+H)⁺.

### Example 22

### 4-(5-(pyridin-3-yl)-1,3,4-oxadiazol-2-yl)benzonitrile trifluoroacetate

Prepared according to Method B. ¹H NMR (500 MHz, DMSO-d₆) δ 7.74 (dd, J=8.2, 4.9 Hz, 1 H), 8.11 (d, J=8.5 Hz, 2 H), 8.35 (d, J=8.5 Hz, 2 H), 8.58 (dt, J=8.2, 1.9 Hz, 1 H), 8.86 (d, J=3.7 Hz, 1 H), 9.35 (d, J=1.2 Hz, 1 H) ppm; MS (DCl/NH₃) m/z 249 (M+H)⁺.

### Example 23

### N,N-dimethyl-3-(5-(pyridin-3-yl)-1,3,4-oxadiazol-2-yl)aniline trifluoroacetate

Prepared according to Method B. ¹H NMR (500 MHz, DMSO-d₆) δ 3.03 (s, 6 H), 7.07 (dt, J=7.6, 2.2 Hz, 1 H), 7.41-7.54 (m, 3 H), 7.73 (dd, J=7.9, 5.2 Hz, 1 H), 8.58 (dt, J=8.0, 1.9 Hz, 1 H), 8.84 (d, J=3.7 Hz, 1 H), 9.34 (s, 1 H) ppm; MS (DCl/NH₃) m/z 267 (M+H)⁺.

### Example 24

### N,N-dimethyl-4-(5-(pyridin-3-yl)-1,3,4-oxadiazol-2-yl)aniline trifluoroacetate

Prepared according to Method B. ¹H NMR (500 MHz, DMSO-d₆) δ 3.04 (s, 6 H), 6.88 (d, J=8.8 Hz, 2 H), 7.73 (dd, J=8.1, 5.0 Hz, 1 H), 7.96 (d, J=9.2 Hz, 2 H), 8.54 (dt, J=7.9, 1.8 Hz, 1 H), 8.82 (dd, J=4.9, 1.2 Hz, 1 H), 9.29 (d, J=1.2 Hz, 1 H) ppm; MS (DCl/NH₃) m/z 267 (M+H)⁺.

### Example 25

### 2-(pyridin-3-yl)-5-(3-(trifluoromethyl)phenyl)-1,3,4-oxadiazole trifluoroacetate

Prepared according to Method B. ¹H NMR (500 MHz, DMSO-d₆) δ 7.73 (dd, J=7.9, 4.9 Hz, 1 H), 7.92 (t, J=7.8 Hz, 1 H), 8.06 (d, J=7.9 Hz, 1 H), 8.44-8.52 (m, 2 H), 8.61 (dt, J=8.0, 1.9 Hz, 1 H), 8.86 (d, J=4.3 Hz, 1 H), 9.38 (s, 1 H) ppm; MS (DCl/NH₃) m/z 292 (M+H)⁺.

### Example 26

### 2-(pyridin-3-yl)-5-(3-(trifluoromethoxy)phenyl)-1,3,4-oxadiazole trifluoroacetate

Prepared according to Method B. ¹H NMR (500 MHz, DMSO-d₆) δ 7.70 (dt, J=8.3, 1.2 Hz, 1 H), 7.74 (dd, J=7.8, 4.7 Hz, 1 H), 7.81 (t, J=8.1 Hz, 1 H), 8.13 (s, 1 H), 8.22 (d, J=7.9 Hz, 1 H), 8.60 (dt, J=7.9, 1.8 Hz, 1 H), 8.86 (d, J=3.7 Hz, 1 H), 9.37 (s, 1 H) ppm; MS (DCl/NH₃) m/z 308 (M+H)⁺.

### Example 27

### 2-(4-phenoxyphenyl)-5-(pyridin-3-yl)-1,3,4-oxadiazole trifluoroacetate

Prepared according to Method B. ¹H NMR (500 MHz, DMSO-d₆) δ 7.16 (d, J=7.6 Hz, 2 H), 7.21 (d, J=8.8 Hz, 2 H), 7.28 (t, J=7.5 Hz, 1 H), 7.47-7.54 (m, 2 H), 7.73 (dd, J=7.9, 4.9 Hz, 1 H), 8.18 (d, J=8.8 Hz, 2 H), 8.55 (dt, J=7.9, 1.8 Hz, 1 H), 8.84 (d, J=3.7 Hz, 1 H), 9.32 (s, 1 H) ppm; MS (DCl/NH₃) m/z 316 (M+H)⁺.

### Example 28

### 2-(4-(benzyloxy)phenyl)-5-(pyridin-3-yl)-1,3,4-oxadiazole trifluoroacetate

Prepared according to Method B. ¹H NMR (500 MHz, DMSO-d₆) δ 5.24 (s, 2 H), 7.34-7.40 (m, 1 H), 7.43 (t, J=7.5 Hz, 2 H), 7.47-7.53 (m, 3 H), 7.66-7.74 (m, 2 H), 8.12 (d, J=8.8 Hz, 2 H), 8.52 (dt, J=7.9, 2.0 Hz, 1 H), 8.82 (d, J=3.1 Hz, 1 H), 9.30 (s, 1 H) ppm; MS (DCl/NH₃) m/z 330 (M+H)⁺.

### Example 29

### 2-(3,4-dimethylphenyl)-5-(pyridin-3-yl)-1,3,4-oxadiazole trifluoroacetate

Prepared according to Method B. ¹H NMR (500 MHz, DMSO-d₆) δ 2.38 (s, 3 H), 2.58 (s, 3 H), 7.35 (t, J=7.6 Hz, 1 H), 7.47 (d, J=7.3 Hz, 1 H), 7.69 (dd, J=7.9, 4.9 Hz, 1 H), 7.86 (d, J=7.3 Hz, 1 H), 8.49 (dt, J=8.2, 1.9 Hz, 1 H), 8.83 (d, J=4.0 Hz, 1 H), 9.28 (s, 1 H) ppm; MS (DCl/NH₃) m/z 252 (M+H)⁺.

### Example 30

### 2-(3,5-dimethylphenyl)-5-(pyridin-3-yl)-1,3,4-oxadiazole trifluoroacetate

Prepared according to Method B. ¹H NMR (500 MHz, DMSO-d₆) δ 2.40 (s, 6 H), 7.32 (s, 1 H), 7.71 (dd, J=8.8, 4.9 Hz, 1 H), 7.80 (s, 2 H), 8.54 (dt, J=8.2, 1.9 Hz, 1 H), 8.83 (d, J=3.4 Hz, 1 H), 9.32 (d, J=1.5 Hz, 1 H) ppm; MS (DCl/NH₃) m/z 252 (M+H)⁺.

### Example 31

### 2-(2,5-dimethylphenyl)-5-(pyridin-3-yl)-1,3,4-oxadiazole trifluoroacetate

Prepared according to Method B. ¹H NMR (500 MHz, DMSO-d₆) δ 2.39 (s, 3 H), 2.65 (s, 3 H), 7.37 (s, 2 H), 7.74 (dd, J=7.6, 4.9 Hz, 1 H), 7.93 (s, 1 H), 8.56 (dt, J=7.9, 1.8 Hz, 1 H), 8.85 (d, J=4.0 Hz, 1 H), 9.32 (s, 1 H) ppm; MS (DCl/NH₃) m/z 252 (M+H)⁺.

### Example 32

### 2-(2,4-dimethylphenyl)-5-(pyridin-3-yl)-1,3,4-oxadiazole trifluoroacetate

Prepared according to Method B. ¹H NMR (500 MHz, DMSO-d₆) δ 2.38 (s, 3 H), 2.67 (s, 3 H), 7.28 (d, J=8.5 Hz, 1 H), 7.31 (s, 1 H), 7.70 (dd, J=7.3, 4.9 Hz, 1 H), 8.00 (d, J=7.9 Hz, 1 H), 8.50 (dt, J=8.1, 1.9 Hz, 1 H), 8.82 (d, J=4.0 Hz, 1 H), 9.28 (s, 1 H) ppm; MS (DCl/NH₃) m/z 252 (M+H)⁺.

### Example 33

### 2-(3,4-dimethylphenyl)-5-(pyridin-3-yl)-1,3,4-oxadiazole trifluoroacetate

Prepared according to Method B. ¹H NMR (500 MHz, DMSO-d₆) δ 2.34 (s, 3 H), 2.35 (s, 3 H), 7.42 (d, J=7.6 Hz, 1 H), 7.69 (dd, J=7.9, 4.9 Hz, 1 H), 7.89 (dd, J=7.8, 1.7 Hz, 1 H), 7.95 (s, 1 H), 8.51 (dt, J=8.2, 1.9 Hz, 1 H), 8.82 (d, J=3.7 Hz, 1 H), 9.30 (s, 1 H) ppm; MS (DCl/NH₃) m/z 252 (M+H)⁺.

### Example 34

### 2-(2,3-dimethoxyphenyl)-5-(pyridin-3-yl)-1,3,4-oxadiazole trifluoroacetate

Prepared according to Method B. ¹H NMR (500 MHz, DMSO-d₆) δ 3.91 (s, 3 H), 3.93 (s, 3 H), 7.30-7.41 (m, 2 H), 7.59 (dd, J=7.8, 1.7 Hz, 1 H), 7.74 (dd, J=7.9, 4.9 Hz, 1 H), 8.52 (dt, J=7.9, 1.8 Hz, 1 H), 8.85 (d, J=4.3 Hz, 1 H), 9.28 (s, 1 H) ppm; MS (DCl/NH₃) m/z 284 (M+H)⁺.

### Example 35

### 2-(2,4-dimethoxyphenyl)-5-(pyridin-3-yl)-1,3,4-oxadiazole trifluoroacetate

Prepared according to Method B. ¹H NMR (500 MHz, DMSO-d₆) δ 3.89 (s, 3 H), 3.95 (s, 3 H), 6.73-6.85 (m, 2 H), 7.74 (dd, J=7.8, 5.0 Hz, 1 H), 7.96 (d, J=8.8 Hz, 1 H), 8.51 (dt, J=8.0, 1.9 Hz, 1 H), 8.84 (d, J=4.0 Hz, 1 H), 9.26 (s, 1 H) ppm; MS (DCl/NH₃) m/z 284 (M+H)⁺.

### Example 36

### 2-(2,5-dimethoxyphenyl)-5-(pyridin-3-yl)-1,3,4-oxadiazole trifluoroacetate

Prepared according to Method B. ¹H NMR (500 MHz, DMSO-d₆) δ 3.82 (s, 3 H), 3.90 (s, 3 H), 7.24-7.27 (m, 2 H), 7.53 (d, J=2.4 Hz, 1 H), 7.71 (dd, J=7.9, 4.9 Hz, 1 H), 8.50 (dt, J=8.0, 1.9 Hz, 1 H), 8.83 (s, 1 H), 9.27 (s, 1 H) ppm; MS (DCl/NH₃) m/z 284 (M+H)⁺.

### Example 37

### 2-(2,4-dimethoxyphenyl)-5-(pyridin-3-yl)-1,3,4-oxadiazole trifluoroacetate

Prepared according to Method B. ¹H NMR (500 MHz, DMSO-d₆) δ 3.88 (s, 3 H), 3.90 (s, 3 H), 7.22 (d, J=8.5 Hz, 1 H), 7.66 (d, J=1.8 Hz, 1 H), 7.70 (dd, J=7.9, 4.9 Hz, 1 H), 7.78 (dd, J=8.4, 2.0 Hz, 1 H), 8.54 (dt, J=8.1, 1.7 Hz, 1 H), 8.83 (d, J=4.0 Hz, 1 H), 9.33 (d, J=1.5 Hz, 1 H) ppm; MS (DCl/NH₃) m/z 284 (M+H)⁺.

### Example 38

### 2-(3,5-dimethoxyphenyl)-5-(pyridin-3-yl)-1,3,4-oxadiazole trifluoroacetate

Prepared according to Method B. ¹H NMR (500 MHz, DMSO-d₆) δ 3.87 (s, 6 H), 6.79 (t, J=2.3 Hz, 1 H), 7.30 (d, J=2.4 Hz, 2 H), 7.71 (dd, J=8.1, 5.0 Hz, 1 H), 8.56 (dt, J=8.1, 1.7 Hz, 1 H), 8.84 (d, J=4.0 Hz, 1 H), 9.35 (s, 1 H) ppm; MS (DCl/NH₃) m/z 284 (M+H)⁺.

### Example 39

### 2-(benzo[d][1,3]dioxol-5-yl)-5-(pyridin-3-yl)-1,3,4-oxadiazole trifluoroacetate

Prepared according to Method B. ¹H NMR (500 MHz, DMSO-d₆) δ 6.18 (s, 2 H), 7.18 (d, J=8.2 Hz, 1 H), 7.66 (d, J=1.5 Hz, 1 H), 7.70 (dd, J=7.9, 4.9 Hz, 1 H), 7.75 (dd, J=8.1, 1.7 Hz, 1 H), 8.53 (dt, J=8.2, 1.9 Hz, 1 H), 8.82 (d, J=4.9 Hz, 1 H), 9.31 (d, J=1.5 Hz, 1 H) ppm; MS (DCl/NH₃) m/z 268 (M+H)⁺.

### Example 40

### 2-(pyridin-3-yl)-5-(3,4,5-trimethoxyphenyl)-1,3,4-oxadiazole trifluoroacetate

Prepared according to Method B. ¹H NMR (500 MHz, DMSO-d₆) δ 3.78 (s, 3 H), 3.93 (s, 6 H), 7.45 (s, 2 H), 7.72 (dd, J=7.9, 4.9 Hz, 1 H), 8.59 (dt, J=8.0, 1.9 Hz, 1 H), 8.84 (dd, J=4.9, 1.2 Hz, 1 H), 9.37 (d, J=1.8 Hz, 1 H) ppm; MS (DCl/NH₃) m/z 314 (M+H)⁺.

### Example 41

### 2-(3,4-dichlorophenyl)-5-(pyridin-3-yl)-1,3,4-oxadiazole trifluoroacetate

Prepared according to Method B. ¹H NMR (500 MHz, DMSO-d₆) δ 7.65 (t, J=8.1 Hz, 1 H), 7.72 (dd, J=7.6, 4.6 Hz, 1 H), 7.96 (dd, J=8.2, 1.5 Hz, 1 H), 8.14 (dd, J=7.9, 1.5 Hz, 1 H), 8.52 (dt, J=8.1, 1.9 Hz, 1 H), 8.85 (d, J=3.4 Hz, 1 H), 9.29 (d, J=1.5 Hz, 1 H) ppm; MS (DCl/NH₃) m/z 294 (M+H)⁺, 292 (M+H)⁺.

### Example 42

### 2-(2,4-dichlorophenyl)-5-(pyridin-3-yl)-1,3,4-oxadiazole trifluoroacetate

Prepared according to Method B. ¹H NMR (500 MHz, DMSO-d₆) δ 7.67-7.76 (m, 2 H), 7.95 (d, J=2.1 Hz, 1 H), 8.21 (d, J=8.2 Hz, 1 H), 8.50 (dt, J=7.9, 1.8 Hz, 1 H), 8.85 (d, J=3.7 Hz, 1 H), 9.28 (d, J=1.2 Hz, 1 H) ppm; MS (DCl/NH₃) m/z 294 (M+H)⁺, 292 (M+H)⁺.

### Example 43

### 2-(2,5-dichlorophenyl)-5-(pyridin-3-yl)-1,3,4-oxadiazole trifluoroacetate

Prepared according to Method B. ¹H NMR (500 MHz, DMSO-d₆) δ 7.72 (dd, J=7.9, 4.9 Hz, 1 H), 7.74-7.81 (m, 2 H), 8.26 (d, J=2.4 Hz, 1 H), 8.54 (dt, J=8.0, 1.9 Hz, 1 H), 8.85 (d, J=3.7 Hz, 1 H), 9.32 (s, 1 H) ppm; MS (DCl/NH₃) m/z 294 (M+H)⁺, 292 (M+H)⁺.

### Example 44

### 2-(3,4-dichlorophenyl)-5-(pyridin-3-yl)-1,3,4-oxadiazole trifluoroacetate

Prepared according to Method B. ¹H NMR (500 MHz, DMSO-d₆) δ 7.70 (dd, J=7.9, 4.9 Hz, 1 H), 7.92 (d, J=8.5 Hz, 1 H), 8.16 (dd, J=8.4, 2.0 Hz, 1 H), 8.42 (d, J=1.8 Hz, 1 H), 8.56 (dt, J=8.1, 1.9 Hz, 1 H), 8.84 (dd, J=4.6, 1.2 Hz, 1 H), 9.35 (d, J=1.2 Hz, 1 H) ppm; MS (DCl/NH₃) m/z 294 (M+H)⁺, 292 (M+H)⁺.

### Example 45

### 5-methyl-2-(5-(pyridin-3-yl)-1,3,4-oxadiazol-2-yl)phenol trifluoroacetate

Prepared according to Method B. ¹H NMR (500 MHz, DMSO-d₆) δ 2.35 (s, 3 H), 6.92 (d, J=7.9 Hz, 1 H), 6.95 (s, 1 H), 7.70 (dd, J=7.6, 5.2 Hz, 1 H), 7.87 (d, J=7.9 Hz, 1 H), 8.49 (dt, J=8.2, 1.8, 1.5 Hz, 1 H), 8.83 (d, J=3.7 Hz, 1 H), 9.27 (s, 1 H) ppm; MS (DCl/NH₃) m/z 254 (M+H)⁺.

### Example 46

### 2-methyl-5-(5-(pyridin-3-yl)-1,3,4-oxadiazol-2-yl)phenol trifluoroacetate

Prepared according to Method B. ¹H NMR (500 MHz, DMSO-d₆) δ 2.23 (s, 3 H), 7.35 (d, J=7.6 Hz, 1 H), 7.50-7.58 (m, 2 H), 7.70 (dd, J=8.2, 4.9 Hz, 1 H), 8.49 (dt, J=8.1, 1.9 Hz, 1 H), 8.82 (d, J=3.7 Hz, 1 H), 9.27 (d, J=1.2 Hz, 1 H) ppm; MS (DCl/NH₃) m/z 254 (M+H)⁺.

### Example 47

### 2-(3-fluoro-2-methylphenyl)-5-(pyridin-3-yl)-1,3,4-oxadiazole trifluoroacetate

Prepared according to Method B. ¹H NMR (500 MHz, DMSO-d₆) δ 2.62 (d, J=2.1 Hz, 3 H), 7.38-7.58 (m, 2 H), 7.71 (dd, J=7.9, 4.9 Hz, 1 H), 7.96 (d, J=7.0 Hz, 1 H), 8.52 (dt, J=8.1, 1.7 Hz, 1 H), 8.84 (d, J=3.7 Hz, 1 H), 9.30 (s, 1 H) ppm; MS (DCl/NH₃) m/z 256 (M+H)⁺.

### Example 48

### 2-(5-fluoro-2-methylphenyl)-5-(pyridin-3-yl)-1,3,4-oxadiazole trifluoroacetate

Prepared according to Method B. ¹H NMR (500 MHz, DMSO-d₆) δ 2.68 (s, 3 H), 7.40 (td, J=8.5, 2.9 Hz, 1 H), 7.53 (dd, J=8.5, 5.8 Hz, 1 H), 7.70 (dd, J=7.3, 4.9 Hz, 1 H), 7.94 (dd, J=9.5, 2.7 Hz, 1 H), 8.53 (dt, J=8.0, 1.9 Hz, 1 H), 8.83 (d, J=3.7 Hz, 1 H), 9.32 (s, 1 H) ppm; MS (DCl/NH₃) m/z 256 (M+H)⁺.

### Example 49

### 2-(3-fluoro-4-methylphenyl)-5-(pyridin-3-yl)-1,3,4-oxadiazole trifluoroacetate

Prepared according to Method B. ¹H NMR (500 MHz, DMSO-d₆) δ 2.35 (d, J=1.2 Hz, 1 H), 7.58 (t, J=7.6 Hz, 1 H), 7.70 (dd, J=7.9, 4.9 Hz, 1 H), 7.91 (d, J=8.8 Hz, 2 H), 8.54 (dt, J=8.0, 1.9 Hz, 1 H), 8.83 (dd, J=4.9, 1.2 Hz, 1 H), 9.32 (d, J=1.5 Hz, 1 H) ppm; MS (DCl/NH₃) m/z 256 (M+H)⁺.

### Example 50

### 2-(2,3-difluorophenyl)-5-(pyridin-3-yl)-1,3,4-oxadiazole trifluoroacetate

Prepared according to Method B. ¹H NMR (500 MHz, DMSO-d₆) δ 7.45-7.55 (m, 1 H), 7.70-7.82 (m, 2 H), 8.02 (dd, J=7.8, 6.3 Hz, 1 H), 8.55 (dt, J=8.1, 2.0, 1.8 Hz, 1 H), 8.86 (dd, J=5.0, 1.7 Hz, 1 H), 9.31 (d, J=1.5 Hz, 1 H) ppm; MS (DCl/NH₃) m/z 260 (M+H)⁺.

### Example 51

### 2-(2,4-difluorophenyl)-5-(pyridin-3-yl)-1,3,4-oxadiazole trifluoroacetate

Prepared according to Method B. ¹H NMR (500 MHz, DMSO-d₆) δ 7.39 (td, J=8.4, 2.1 Hz, 1 H), 7.58 (ddd, J=11.1, 9.0, 2.4 Hz, 1 H), 7.75 (dd, J=8.1, 5.0 Hz, 1 H), 8.28 (td, J=8.5, 6.4 Hz, 1 H), 8.55 (dt, J=8.0, 1.9 Hz, 1 H), 8.86 (dd, J=4.9, 1.5 Hz, 1 H), 9.30 (d, J=1.5 Hz, 1 H) ppm; MS (DCl/NH₃) m/z 260 (M+H)⁺

### Example 52

### 2-(2,5-difluorophenyl)-5-(pyridin-3-yl)-1,3,4-oxadiazole trifluoroacetate

Prepared according to Method B. ¹H NMR (500 MHz, DMSO-d₆) δ 7.55-7.62 (m, 2 H), 7.76 (dd, J=7.9, 4.9 Hz, 1 H), 7.97-8.18 (m, 1 H), 8.58 (dt, J=8.0, 1.9 Hz, 1 H), 8.87 (dd, J=4.9, 1.5 Hz, 1 H), 9.33 (d, J=1.8 Hz, 1 H) ppm; MS (DCl/NH₃) m/z 260 (M+H)⁺.

### Example 53

### 2-(3,5-difluorophenyl)-5-(pyridin-3-yl)-1,3,4-oxadiazole trifluoroacetate

Prepared according to Method B. ¹H NMR (500 MHz, DMSO-d₆) δ 7.52-7.63 (m, 1 H), 7.74 (dd, J=7.9, 4.9 Hz, 1 H), 7.83-7.94 (m, 2 H), 8.60 (dt, J=8.0, 1.9 Hz, 1 H), 8.86 (dd, J=5.0, 1.4 Hz, 1 H), 9.36 (d, J=1.5 Hz, 1 H) ppm; MS (DCl/NH₃) m/z 260 (M+H)⁺.

### Example 54

### 1-(4-(5-(pyridin-3-yl)-1,3,4-oxadiazol-2-yl)phenyl)ethanone trifluoroacetate

Prepared according to Method B. ¹H NMR (500 MHz, DMSO-d₆) δ 2.67 (s, 3 H), 7.72 (dd, J=8.2, 4.9 Hz, 1 H), 8.20 (d, J=8.5 Hz, 2 H), 8.32 (d, J=8.2 Hz, 2 H), 8.56 (dt, J=7.9. 1.8 Hz, 1 H), 8.85 (d, J=3.4 Hz, 1 H), 9.34 (d, J=1.5 Hz, 1 H) ppm; MS (DCl/NH₃) m/z 266 (M+H)⁺.

### Example 55

### 2-(4-isopropylphenyl)-5-(pyridin-3-yl)-1,3,4-oxadiazole trifluoroacetate

Prepared according to Method B. ¹H NMR (500 MHz, DMSO-d₆) δ 1.26 (d, J=7.0 Hz, 6 H), 2.85-3.18 (m, 1 H), 7.53 (d, J=8.2 Hz, 2 H), 7.74 (dd, J=7.3, 4.9 Hz, 1 H), 8.10 (d, J=8.5 Hz, 2 H), 8.57 (dt, J=8.3, 1.8 Hz, 1 H), 8.85 (dd, J=5.0, 1.7 Hz, 1 H), 9.33 (d, J=1.5 Hz, 1 H) ppm; MS (DCl/NH₃) m/z 266 (M+H)⁺.

### Example 56

### 2-(3-methoxy-4-methylphenyl)-5-(pyridin-3-yl)-1,3,4-oxadiazole trifluoroacetate

Prepared according to Method B. ¹H NMR (500 MHz, DMSO-d₆) δ 2.26 (s, 3 H), 3.94 (s, 3 H), 7.42 (d, J=7.9 Hz, 1 H), 7.63 (s, 1 H), 7.66-7.73 (m, 2 H), 8.54 (dt, J=8.2, 1.9 Hz, 1 H), 8.83 (d, J=3.4 Hz, 1 H), 9.33 (s, 1 H) ppm; MS (DCl/NH₃) m/z 268 (M+H)⁺.

### Example 57

### 2-(4-ethoxyphenyl)-5-(pyridin-3-yl)-1,3,4-oxadiazole trifluoroacetate

Prepared according to Method B. ¹H NMR (500 MHz, DMSO-d₆) δ 1.38 (t, J=6.9 Hz, 3 H), 4.15 (q, J=7.0 Hz, 2 H), 7.17 (d, J=8.8 Hz, 2 H), 7.74 (dd, J=7.9, 4.9 Hz, 1 H), 8.10 (d, J=8.8 Hz, 2 H), 8.55 (dt, J=7.9, 1.8 Hz, 1 H), 8.84 (dd, J=4.9, 1.2 Hz, 1 H), 9.31 (d, J=1.5 Hz, 1 H) ppm; MS (DCl/NH₃) m/z 268 (M+H)⁺.

### Example 58

### 2-(4-(methylthio)phenyl)-5-(pyridin-3-yl)-1,3,4-oxadiazole trifluoroacetate

Prepared according to Method B. ¹H NMR (500 MHz, DMSO-d₆) δ 2.57 (s, 3 H), 7.50 (d, J=8.5 Hz, 2 H), 7.71 (dd, J=8.2, 5.5 Hz, 1 H), 8.09 (d, J=8.5 Hz, 2 H), 8.53 (dt, J=7.9, 2.0 Hz, 1 H), 8.83 (d, J=3.7 Hz, 1 H), 9.31 (d, J=1.5 Hz, 1 H) ppm; MS (DCl/NH₃) m/z 270 (M+H)⁺.

### Example 59

### 2-(3-fluoro-4-methoxyphenyl)-5-(pyridin-3-yl)-1,3,4-oxadiazole trifluoroacetate

Prepared according to Method B. ¹H NMR (500 MHz, DMSO-d₆) δ 3.97 (s, 3 H), 7.43 (t, J=9.0 Hz, 1 H), 7.69 (dd, J=7.6, 5.2 Hz, 1 H), 7.96-8.07 (m, 2 H), 8.52 (dt, J=8.2, 1.9 Hz, 1 H), 8.82 (d, J=5.2 Hz, 1 H), 9.31 (s, 1 H) ppm; MS (DCl/NH₃) m/z 272 (M+H)⁺.

### Example 60

### 2-(naphthalen-1-yl)-5-(pyridin-3-yl)-1,3,4-oxadiazole trifluoroacetate

Prepared according to Method B. ¹H NMR (500 MHz, DMSO-d₆) δ 7.62-7.90 (m, 4 H), 8.14 (d, J=8.2 Hz, 1 H), 8.27 (d, J=8.2 Hz, 1 H), 8.46 (dd, J=7.3, 1.2 Hz, 1 H), 8.61 (dt, J=7.9, 1.8 Hz, 1 H), 8.87 (d, J=4.3 Hz, 1 H), 9.17 (d, J=8.5 Hz, 1 H), 9.38 (s, 1 H) ppm; MS (DCl/NH₃) m/z 274 (M+H)⁺.

### Example 61

### 2-(naphthalen-2-yl)-5-(pyridin-3-yl)-1,3,4-oxadiazole trifluoroacetate

Prepared according to Method B. ¹H NMR (500 MHz, DMSO-d₆) δ 7.67-7.80 (m, 3 H), 8.03-8.11 (m, 1 H), 8.18 (d, J=8.2 Hz, 2 H), 8.23 (dd, J=8.5, 1.5 Hz, 1 H), 8.60 (dt, J=8.0, 1.9 Hz, 1 H), 8.82 (s, 1 H), 8.86 (d, J=3.7 Hz, 1 H), 9.38 (s, 1 H) ppm; MS (DCl/NH₃) m/z 274 (M+H)⁺.

### Example 62

### 4-chloro-2-(5-(pyridin-3-yl)-1,3,4-oxadiazol-2-yl)phenol trifluoroacetate

Prepared according to Method B. ¹H NMR (500 MHz, DMSO-d₆) δ 7.16 (d, J=8.8 Hz, 1 H), 7.55 (dd, J=8.8, 2.7 Hz, 1 H), 7.70 (dd, J=8.1, 4.7 Hz, 1 H), 8.01 (d, J=2.7 Hz, 1 H), 8.51 (dt, J=8.1, 1.7 Hz, 1 H), 8.83 (dd, J=4.9, 1.5 Hz, 1 H), 9.29 (d, J=1.5 Hz, 1 H) ppm; MS (DCl/NH₃) m/z 276 (M+H)⁺, 274 (M+H)⁺.

### Example 63

### 2-(4-tert-butylphenyl)-5-(pyridin-3-yl)-1,3,4-oxadiazole trifluoroacetate

Prepared according to Method B. ¹H NMR (500 MHz, DMSO-d₆) δ 1.35 (s, 9 H), 7.68 (d, J=8.5 Hz, 2 H), 7.74 (dd, J=7.9, 4.9 Hz, 1 H), 8.10 (d, J=8.5 Hz, 2 H), 8.57 (dt, J=8.0, 1.9 Hz, 1 H), 8.85 (d, J=4.3 Hz, 1 H), 9.33 (s, 1 H) ppm; MS (DCl/NH₃) m/z 280 (M+H)⁺.

### Example 64

### N-(4-(5-(pyridin-3-yl)-1,3,4-oxadiazol-2-yl)phenyl)acetamide trifluoroacetate

Prepared according to Method B. ¹H NMR (500 MHz, DMSO-d₆) δ 2.12 (s, 3 H), 7.69 (dd, J=8.2, 4.6 Hz, 1 H), 7.83 (d, J=8.8 Hz, 2 H), 8.12 (d, J=8.8 Hz, 2 H), 8.51 (dt, J=8.2, 1.9 Hz, 1 H), 8.82 (dd, J=4.9, 1.5 Hz, 1 H), 9.30 (d, J=2.4 Hz, 1 H) ppm; MS (DCl/NH₃) m/z 281 (M+H)⁺.

### Example 65

### 2-(4-propoxyphenyl)-5-(pyridin-3-yl)-1,3,4-oxadiazole trifluoroacetate

Prepared according to Method B. ¹H NMR (500 MHz, DMSO-d₆) δ 1.01 (t, J=7.3 Hz, 3 H), 1.68-1.93 (m, 2 H), 4.06 (t, J=6.6 Hz, 2 H), 7.18 (d, J=8.8 Hz, 2 H), 7.69 (dd, J=8.2, 4.9 Hz, 1 H), 8.11 (d, J=8.8 Hz, 2 H), 8.51 (dt, J=8.2, 1.9 Hz, 1 H), 8.82 (dd, J=5.0, 1.4 Hz, 1 H), 9.29 (d, J=1.5 Hz, 1 H) ppm; MS (DCl/NH₃) m/z 282 (M+H)⁺.

### Example 66

### 2-(4-isopropoxyphenyl)-5-(pyridin-3-yl)-1,3,4-oxadiazole trifluoroacetate

Prepared according to Method B. ¹H NMR (500 MHz, DMSO-d₆) δ 1.33 (d, J=5.8 Hz, 6 H), 4.64-4.88 (m, 1 H), 7.16 (d, J=8.8 Hz, 2 H), 7.73 (dd, J=8.1, 5.0 Hz, 1 H), 8.09 (d, J=9.2 Hz, 2 H), 8.55 (dt, J=8.2, 1.9 Hz, 1 H), 8.83 (dd, J=4.9, 1.2 Hz, 1 H), 9.31 (d, J=1.8 Hz, 1 H) ppm; MS (DCl/NH₃) m/z 282 (M+H)⁺.

### Example 67

### 2-(5-chloro-2-methoxyphenyl)-5-(pyridin-3-yl)-1,3,4-oxadiazole trifluoroacetate

Prepared according to Method B. ¹H NMR (500 MHz, DMSO-d₆) δ 3.96 (s, 3 H), 7.35 (d, J=9.2 Hz, 1 H), 7.69 (dd, J=8.8, 2.7 Hz, 1 H), 7.73 (dd, J=7.9, 4.9 Hz, 1 H), 8.05 (d, J=2.7 Hz, 1 H), 8.54 (dt, J=7.9, 1.8 Hz, 1 H), 8.85 (d, J=3.4 Hz, 1 H), 9.30 (d, J=1.5 Hz, 1 H) ppm; MS (DCl/NH₃) m/z 288 (M+H)⁺.

### Example 68

### 2-(4-fluoronaphthalen-1-yl)-5-(pyridin-3-yl)-1,3,4-oxadiazole trifluoroacetate

Prepared according to Method B. ¹H NMR (500 MHz, DMSO-d₆) δ 7.62 (dd, J=10.1, 8.2 Hz, 1 H), 7.72 (dd, J=7.8, 4.4 Hz, 1 H), 7.84 (t, J=7.3 Hz, 1 H), 7.92 (ddd, J=8.5, 7.0, 1.2 Hz, 1 H), 8.27 (d, J=8.2 Hz, 1 H), 8.49 (dd, J=8.2, 5.5 Hz, 1 H), 8.58 (dt, J=7.9, 2.0 Hz, 1 H), 8.85 (dd, J=4.6, 1.5 Hz, 1 H), 9.24 (d, J=8.5 Hz, 1 H), 9.36 (d, J=1.8 Hz, 1 H) ppm; MS (DCl/NH₃) m/z 292 (M+H)⁺.

### Example 69

### N,N-diethyl-4-(5-(pyridin-3-yl)-1,3,4-oxadiazol-2-yl)aniline trifluoroacetate

Prepared according to Method B. ¹H NMR (500 MHz, DMSO-d₆) δ 1.15 (t, J=7.0 Hz, 6 H), 3.45 (q, J=7.0 Hz, 4 H), 6.88 (d, J=9.2 Hz, 2 H), 7.73 (dd, J=7.8, 4.7 Hz, 1 H), 7.94 (d, J=9.2 Hz, 2 H), 8.54 (dt, J=7.9, 1.8 Hz, 1 H), 8.82 (dd, J=4.9, 1.5 Hz, 1 H), 9.29 (d, J=1.5 Hz, 1 H) ppm; MS (DCl/NH₃) m/z 295 (M+H)⁺.

### Example 70

### 2-(4-butoxyphenyl)-5-(pyridin-3-yl)-1,3,4-oxadiazole trifluoroacetate

Prepared according to Method B. ¹H NMR (500 MHz, DMSO-d₆) δ 0.95 (t, J=7.3 Hz, 3 H), 1.35-1.59 (m, 2 H), 1.65-1.91 (m, 2 H), 4.10 (t, J=6.4 Hz, 2 H), 7.18 (d, J=9.2 Hz, 2 H), 7.68 (dd, J=7.9, 4.9 Hz, 1 H), 8.10 (d, J=9.2 Hz, 2 H), 8.50 (dt, J=7.9, 2.0 Hz, 1 H), 8.81 (dd, J=4.7, 1.4 Hz, 1 H), 9.29 (d, J=1.8 Hz, 1 H) ppm; MS (DCl/NH₃) m/z 296 (M+H)⁺.

### Example 71

### 2-(2-methoxy-4-(methylthio)phenyl)-5-(pyridin-3-yl)-1,3,4-oxadiazole trifluoroacetate

Prepared according to Method B. ¹H NMR (500 MHz, DMSO-d₆) δ 2.58 (s, 3 H), 3.98 (s, 3 H), 7.05 (dd, J=8.2, 1.8 Hz, 1 H), 7.08 (d, J=1.8 Hz, 1 H), 7.72 (dd, J=7.3, 4.9 Hz, 1 H), 7.95 (d, J=8.2 Hz, 1 H), 8.49 (dt, J=8.1, 2.0, 1.8 Hz, 1 H), 8.83 (d, J=3.7 Hz, 1 H), 9.26 (s, 1 H) ppm; MS (DCl/NH₃) m/z 300 (M+H)⁺.

### Example 72

### 2-(4-(methylsulfonyl)phenyl)-5-(pyridin-3-yl)-1,3,4-oxadiazole trifluoroacetate

Prepared according to Method B. ¹H NMR (500 MHz, DMSO-d₆) δ 3.31 (s, 3 H), 7.71 (dd, J=8.1, 5.6 Hz, 1 H), 8.19 (d, J=8.8 Hz, 2 H), 8.44 (d, J=8.8 Hz, 2 H), 8.56 (dt, J=8.1, 1.9 Hz, 1 H), 8.85 (d, J=4.9 Hz, 1 H), 9.34 (s, 1 H) ppm; MS (DCl/NH₃) m/z 302 (M+H)⁺.

### Example 73

### 2-(2-chloro-5-(methylthio)phenyl)-5-(pyridin-3-yl)-1 3,4-oxadiazole trifluoroacetate

Prepared according to Method B. ¹H NMR (500 MHz, DMSO-d₆) δ 2.58 (s, 3 H), 7.56 (dd, J=8.5, 2.4 Hz, 1 H), 7.68 (d, J=8.5 Hz, 1 H), 7.73 (dd, J=7.9, 4.9 Hz, 1 H), 7.96 (d, J=2.1 Hz, 1 H), 8.54 (dt, J=8.2, 1.9 Hz, 1 H), 8.85 (dd, J=4.9, 1.5 Hz, 1 H), 9.31 (d, J=1.5 Hz, 1 H) ppm; MS (DCl/NH₃) m/z 306 (M+H)⁺, 304 (M+H)⁺.

### Example 74

### 2-(2-fluoro-5-(trifluoromethyl)phenyl)-5-(pyridin-3-yl)-1,3,4-oxadiazole trifluoroacetate

Prepared according to Method B. ¹H NMR (500 MHz, DMSO-d₆) δ 7.71 (dd, J=7.6, 5.2 Hz, 1 H), 7.99-8.07 (m, 2 H), 8.51 (d, J=1.8 Hz, 1 H), 8.54 (dt, J=8.2, 1.9 Hz, 1 H), 8.85 (dd, J=4.9, 1.2 Hz, 1 H), 9.32 (d, J=1.2 Hz, 1 H) ppm; MS (DCl/NH₃) m/z 310 (M+H)⁺.

### Example 75

### 2-(2-chloro-5-(trifluoromethyl)phenyl)-5-(pyridin-3-yl)-1,3,4-oxadiazole trifluoroacetate

Prepared according to Method B. ¹H NMR (500 MHz, DMSO-d₆) δ 7.71 (dd, J=7.6, 5.2 Hz, 1 H), 7.98-8.09 (m, 2 H), 8.51 (d, J=1.8 Hz, 1 H), 8.54 (dt, J=8.2, 1.9 Hz, 1 H), 8.85 (dd, J=4.9, 1.2 Hz, 1 H), 9.32 (d, J=1.2 Hz, 1 H) ppm; MS (DCl/NH₃) m/z 328 (M+H)⁺, 326 (M+H)⁺.

### Example 76

### 2-(2-phenethylphenyl)-5-(pyridin-3-yl)-1,3,4-oxadiazole trifluoroacetate

Prepared according to Method B. ¹H NMR (500 MHz, DMSO-d₆) δ 2.83-2.98 (m, 2 H), 3.32-3.49 (m, 2 H), 7.12-7.19 (m, 1 H), 7.20-7.30 (m, 4 H), 7.45-7.54 (m, 2 H), 7.56-7.61 (m, 1 H), 7.69 (dd, J=7.9, 4.9 Hz, 1 H), 8.08 (d, J=6.4 Hz, 1 H), 8.48 (dt, J=8.2, 1.9 Hz, 1 H), 8.83 (d, J=3.7 Hz, 1 H), 9.27 (s, 1 H) ppm: MS (DCl/NH₃) m/z 328 (M+H)⁺

### Example 77

### 2-(2-bromo-5-methoxyphenyl)-5-(pyridin-3-yl)-1,3,4-oxadiazole trifluoroacetate

Prepared according to Method B. ¹H NMR (500 MHz, DMSO-d₆) δ 3.87 (s, 3 H), 7.21 (dd, J=9.0, 3.2 Hz, 1 H), 7.63 (d, J=3.1 Hz, 1 H), 7.71 (dd, J=7.9, 4.9 Hz, 1 H), 7.81 (d, J=8.8 Hz, 1 H), 8.51 (dt, J=7.9, 2.0 Hz, 1 H), 8.85 (d, J=3.7 Hz, 1 H), 9.29 (d, J=1.5 Hz, 1 H) ppm; MS (DCl/NH₃) m/z 334 (M+H)⁺, 332 (M+H)⁺.

### Example 78

### 2-(5-bromo-2-chlorophenyl)-5-(pyridin-3-yl)-1,3,4-oxadiazole trifluoroacetate

Prepared according to Method B. ¹H NMR (500 MHz, DMSO-d₆) δ 7.65-7.75 (m, 2 H), 7.88 (dd, J=8.5, 2.4 Hz, 1 H), 8.37 (d, J=2.4 Hz, 1 H), 8.54 (dt, J=8.2, 1.9 Hz, 1 H), 8.85 (d, J=3.4 Hz, 1 H), 9.32 (s, 1 H) ppm; MS (DCl/NH₃) m/z 338 (M+H)⁺, 336 (M+H)⁺.

### Example 79

### 2-(2-iodophenyl)-5-(pyridin-3-yl)-1,3,4-oxadiazole trifluoroacetate

Prepared according to Method B. ¹H NMR (500 MHz, DMSO-d₆) δ 7.42 (td, J=7.8, 1.5 Hz, 1 H), 7.67 (t, J=7.6 Hz, 1 H), 7.71 (dd, J=7.9, 4.9 Hz, 1 H), 8.00 (dd, J=7.9, 1.5 Hz, 1 H), 8.18 (d, J=7.6 Hz, 1 H), 8.50 (dt, J=8.1, 2.0, 1.8 Hz, 1 H), 8.85 (dd, J=4.9, 1.5 Hz, 1 H), 9.28 (d, J=1.8 Hz, 1 H) ppm; MS (DCl/NH₃) m/z 350 (M+H)⁺.

### Example 80

### 2-(3-iodophenyl)-5-(pyridin-3-yl)-1,3,4-oxadiazole trifluoroacetate

Prepared according to Method B. ¹H NMR (500 MHz, DMSO-d₆) δ 7.46 (t, J=7.8 Hz, 1 H), 7.70 (dd, J=8.2, 4.9 Hz, 1 H), 8.05 (d, J=7.9 Hz, 1 H), 8.20 (d, J=7.6 Hz, 1 H), 8.50 (t, J=1.7 Hz, 1 H), 8.56 (dt, J=8.1, 1.7 Hz, 1 H), 8.84 (d, J=4.3 Hz, 1 H), 9.34 (s, 1 H) ppm; MS (DCl/NH₃) m/z 350 (M+H)⁺.

### Example 81

### 2-(4-iodophenyl)-5-(pyridin-3-yl)-1,3,4-oxadiazole trifluoroacetate

Prepared according to Method B. ¹H NMR (500 MHz, DMSO-d₆) δ 7.69 (dd, J=8.1, 5.0 Hz, 1 H), 7.96 (d, J=8.5 Hz, 2 H), 8.05 (d, J=8.5 Hz, 2 H), 8.52 (dt, J=8.0, 1.9 Hz, 1 H), 8.83 (d, J=3.7 Hz, 1 H), 9.31 (s, 1 H) ppm; MS (DCl/NH₃) m/z 350 (M+H)⁺.

### Example 82

### 2-(pyridin-3-yl)-5-(pyrimidin-5-yl)-1,3,4-oxadiazole trifluoroacetate

Prepared according to Method B. ¹H NMR (500 MHz, DMSO-d₆) δ 7.68-7.76 (m, 1 H), 8.50-8.61 (m, 1 H), 8.81-8.89 (m, 1 H), 8.90-8.98 (m, 1 H), 9.28-9.38 (m, 1 H), 9.50 (s, 1 H), 9.67 (s, 1 H) ppm; MS (DCl/NH₃) m/z 226 (M+H)⁺.

### Example 83

### 2-(5-methylpyrazin-2-yl)-5-(pyridin-3-yl)-1,3,4-oxadiazole trifluoroacetate

Prepared according to Method B. ¹H NMR (500 MHz, DMSO-d₆) δ 2.66 (s, 3 H), 7.72 (dd, J=7.3, 4.9 Hz, 1 H), 8.53 (dt, J=8.1, 2.0, 1.8 Hz, 1 H), 8.80 (s, 1 H), 8.85 (dd, J=4.9, 1.5 Hz, 1 H), 9.31 (d, J=1.5 Hz, 1 H), 9.35 (d, J=1.5 Hz, 1 H) ppm; MS (DCl/NH₃) m/z 240 (M+H)⁺.

### Example 84

### 2-(2-chloro-6-methylpyridin-3-yl)-5-(pyridin-3-yl)-1,3,4-oxadiazole

Prepared according to Method B. ¹H NMR (500 MHz, DMSO-d₆) δ 2.59 (s, 3 H), 7.57 (d, J=7.9 Hz, 1 H), 7.65-7.76 (m, 1 H), 8.46-8.54 (m, 2 H), 8.85 (s, 1 H), 9.29 (s, 1 H) ppm; MS (DCl/NH₃) m/z 275 (M+H)⁺, 273 (M+H)⁺.

### Example 85

### 2-(2-methyl-6-(trifluoromethyl)pyridin-3-yl)-5-(pyridin-3-yl)-1,3,4-oxadiazole

Prepared according to Method B. ¹H NMR (500 MHz, DMSO-d₆) δ 3.00 (s, 3 H), 7.71 (dd, J=7.9, 4.9 Hz, 1 H), 8.01 (d, J=8.2 Hz, 1 H), 8.54 (d, J=7.9 Hz, 1 H), 8.77 (d, J=7.9 Hz, 1 H), 8.86 (s, 1 H), 9.33 (s, 1 H) ppm; MS (DCl/NH₃) m/z 307 (M+H)⁺.

### Example 86

### 2-(2-(ethylthio)pyridin-3-yl)-5-(pyridin-3-yl)-1,3,4-oxadiazole

Prepared according to Method B. ¹H NMR (500 MHz, DMSO-d₆) δ 1.34 (t, J=7.3 Hz, 3 H), 3.25 (q, J=7.3 Hz, 2 H), 7.40 (dd, J=7.9, 4.9 Hz, 1 H), 7.71 (dd, J=7.6, 4.9 Hz, 1 H), 8.47 (dd, J=7.6, 1.8 Hz, 1 H), 8.52 (dt, J=8.2, 1.9 Hz, 1 H), 8.70 (dd, J=4.7, 1.7 Hz, 1 H), 8.84 (d, J=4.0 Hz, 1 H), 9.31 (s, 1 H) ppm; MS (DCl/NH₃) m/z 285 (M+H)⁺.

### Example 87

### 2-(2,6-dimethoxypyridin-3-yl)-5-(pyridin-3-yl)-1,3,4-oxadiazole

Prepared according to Method B. ¹H NMR (500 MHz, DMSO-d₆) δ 3.99 (s, 3 H), 4.08 (s, 3 H), 6.65 (d, J=8.2 Hz, 1 H), 7.74-7.85 (m, 1 H), 8.36 (d, J=8.5 Hz, 1 H), 8.46 (d, J=7.9 Hz, 1 H), 8.82 (s, 1 H), 9.25 (s, 1 H) ppm; MS (DCl/NH₃) m/z 285 (M+H)⁺.

### Example 88

### 2-(2-(methylthio)pyridin-3-yl)-5-(pyridin-3-yl)-1,3,4-oxadiazole

Prepared according to Method B. ¹H NMR (500 MHz, DMSO-d₆) δ 32.59 (s, 3 H), 7.41 (dd, J=7.8, 4.7 Hz, 1 H), 7.71 (dd, J=7.8, 5.0 Hz, 1 H), 8.49 (dd, J=7.9, 1.8 Hz, 1 H), 8.53 (dt, J=8.2, 1.9 Hz, 1 H), 8.72 (dd, J=4.7, 1.7 Hz, 1 H), 8.84 (d, J=5.2 Hz, 1 H), 9.32 (s, 1 H) ppm; MS (DCl/NH₃) m/z 271 (M+H)⁺.

### Example 89

### 5-chloro-3-(5-(pyridin-3-yl)-1,3,4-oxadiazol-2-yl)pyridin-2-ol

Prepared according to Method B. ¹H NMR (500 MHz, DMSO-d₆) δ 7.69 (dd, J=8.1, 5.0 Hz, 1 H), 7.98 (d, J=2.7 Hz, 1 H), 8.42 (d, J=3.1 Hz, 1 H), 8.49 (dt, J=7.9, 1.8 Hz, 1 H), 8.82 (d, J=4.3 Hz, 1 H), 9.25 [s (broad), 1 H] ppm; MS (DCl/NH₃) m/z 277 (M+H)⁺, 275 (M+H)⁺.

### Example 90

### 2-(2,6-dichloro-5-fluoropyridin-3-yl)-5-(pyridin-3-yl)-1,3,4-oxadiazole

Prepared according to Method B. ¹H NMR (500 MHz, DMSO-d₆) δ 7.72 (dd, J=7.8, 5.0 Hz, 1 H), 8.55 (dt, J=8.0, 1.8 Hz, 1 H), 8.81 (d, J=8.2 Hz, 1 H), 8.86 (d, J=3.7 Hz, 1 H), 9.34 (s, 1 H) ppm; MS (DCl/NH₃) m/z 313 (M+H)⁺, 311 (M+H)⁺.

### Example 91

### 2-(2,5-dichloropyridin-3-yl)-5-(pyridin-3-yl)-1,3,4-oxadiazole

Prepared according to Method B. ¹H NMR (500 MHz, DMSO-d₆) δ 7.72 (dd, J=7.9, 4.9 Hz, 1 H), 8.56 (d, J=7.9 Hz, 1 H), 8.78 (dd, J=2.7 Hz, 1 H), 8.79 (d, J=2.7 Hz, 1 H), 8.86 (s, 1 H), 9.35 (s, 1 H) ppm; MS (DCl/NH₃) m/z 295 (M+H)⁺, 293 (M+H)⁺.

### Example 92

### 2-(6-chloropyridin-3-yl)-5-(pyridin-3-yl)-1,3,4-oxadiazole

Prepared according to Method B. ¹H NMR (500 MHz, DMSO-d₆) δ 7.71 (dd, J=7.9, 4.6 Hz, 1 H), 7.82 (d, J=7.6 Hz, 1 H), 8.55 (dt, J=8.1, 1.9 Hz, 1 H), 8.58 (dd, J=8.4, 2.6 Hz, 1 H), 8.85 (s, 1 H), 9.19 (d, J=1.8 Hz, 1 H), 9.35 (s, 1 H) ppm; MS (DCl/NH₃) m/z 261 (M+H)⁺, 259 (M+H)⁺.

### Example 93

### 2-(2,6-dichloropyridin-3-yl)-5-(pyridin-3-yl)-1,3,4-oxadiazole

Prepared according to Method B. ¹H NMR (500 MHz, DMSO-d₆) δ 7.72 (dd, J=7.9, 4.3 Hz, 1 H), 7.85 (d, J=8.2 Hz, 1 H), 8.52 (d, J=7.9 Hz, 1 H), 8.67 (d, J=8.2 Hz, 1 H), 8.87 (s, 1 H), 9.31 (s, 1 H) ppm; MS (DCl/NH₃) m/z 295 (M+H)⁺, 293 (M+H)⁺.

### Example 94

### 2-(2-chloropyridin-3-yl)-5-(pyridin-3-yl)-1,3,4-oxadiazole

Prepared according to Method B. ¹H NMR (500 MHz, DMSO-d₆) δ 7.72 (dd, J=7.9, 4.9 Hz, 1 H), 7.74 [s (broad), 1 H[, 7.82 [s (broad), 1 H], 8.52 (d, J=7.9 Hz, 1 H), 8.62 (dd, J=7.8, 2.0 Hz, 1 H), 8.69 (dd, J=4.7, 2.0 Hz, 1 H), 8.90 (d, J=7.9 Hz, 1 H) ppm; MS (DCl/NH₃) m/z 261 (M+H)⁺, 259 (M+H)⁺.

### Example 95

### 2-(pyridin-3-yl)-5-(quinolin-3-yl)-1,3,4-oxadiazole

Prepared according to Method B. ¹H NMR (500 MHz, DMSO-d₆) δ 7.73 (dd, J=8.1, 4.7 Hz, 1 H), 7.80 (td, J=7.6, 1.1 Hz, 1 H), 7.97 (ddd, J=8.5, 6.9, 1.4 Hz, 1 H), 8.18 (d, J=8.5 Hz, 1 H), 8.25 (d, J=7.6 Hz, 1 H), 8.60 (td, J=8.2, 1.9 Hz, 1 H), 8.86 (d, 8.86 (d, J=3.1 Hz, 1 H), 9.24 (d, J=2.1 Hz, 1 H), 9.39 (s, 1 H), 9.59 (d, J=2.1 Hz, 1 H) ppm; MS (DCl/NH₃) m/z 275 (M+H)⁺.

### Compositions of the Invention

The invention also provides pharmaceutical compositions comprising a therapeutically effective amount of a compound of formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier,” as used herein, means a non-toxix, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection, and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N. Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The compounds of the invention can be used in the form of pharmaceutically acceptable salts derived from inorganic or organic acids. The term “pharmaceutically acceptable salts,” as used herein, include salts and zwitterions of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine.

The invention also contemplates pharmaceutically acceptable compounds that when administered to a patient in need may be converted through in vivo biotransformation into compounds of formula (I).

### Methods of Use

The biological effects of the compounds of the invention result from positive allosteric modulation of an α4β2 subtype of nicotinic acetylcholine receptor. Representative compounds of the invention demonstrate α4β2 nAChR positive allosteric modulator activity. As such, compounds and compositions of the invention are useful for the treatment of conditions and disorders related to cholinergic dysfunction and for conditions and disorders responsive to the action of nAChR modulators. The method is useful for treating, preventing or both treating and preventing conditions and disorders related to α4β2 nAChR positive allosteric modulator activity, particularly in mammals.

More particularly, the method is useful for conditions and disorders related to attention deficit disorder, attention deficit hyperactivity disorder (ADHD), Alzheimer's disease (AD), schizophrenia, mild cognitive impairment, age-associated memory impairment (AAMI), senile dementia, AIDS dementia, Pick's Disease, dementia associated with Lewy bodies, dementia associated with Down's syndrome, schizophrenia, smoking cessation, substance abuse including alcohol abuse, amyotrophic lateral sclerosis, Huntington's disease, diminished CNS function associated with traumatic brain injury, acute pain, post-surgical pain, chronic pain, inflammatory pain, neuropathic pain, infertility, lack of circulation, need for new blood vessel growth associated with wound healing, more particularly circulation around a vascular occlusion, need for new blood vessel growth associated with vascularization of skin grafts, ischemia, inflammation, sepsis, wound healing, and other complications associated with diabetes, among other systemic and neuroimmunomodulatory activities. The method is useful for conditions and disorders related to conditions and disorders characterized by neuropsychological and cognitive dysfunction, for example in Alzheimer's disease, bipolar disorder, schizophrenia, schizoaffective disorder, and other related disorders characterized by neuropsychological and cognitive dysfunction, in particular.

Compounds of the invention also are useful for treating, preventing or both treating and preventing pain, particularly in mammals. Administration of compounds of the invention is useful for treating nociceptive and neuropathic forms of pain, for example, chronic pain, analgesic pain, post-surgical pain, neuropathic pain, and diabetic neuropathy. Such compounds are particularly beneficial for reducing adverse ganglionic effects such as at gastrointestinal systems (e.g. emesis) and for enhancing the of nAChR ligands in such treatment.

A further aspect of the invention relates to a method of selectively modulating nAChR activity, for example α4β2 nAChR positive allosteric modulator activity, in combination with a nicotinic agonist or partial agonist to improve the tolerability of therapy using such nicotinic agonist or partial agonist, which is further described herein below. When dosed in combination with nAChR agonists, such compounds could enhance efficacy in various disease states including pain and cognitive deficits by preferentially modulating α4β2 activity, and enabling improved separation from potential adverse emesis, cardiovascular and other effects.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in a pharmaceutically acceptable salt. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or animal ranges from about 0.010 mg/kg body weight to about 500 mg/kg body weight. More preferable doses can be in the range of from about 0.10 mg/kg body weight to about 50 mg/kg body weight. If desired, the effective daily dose can be divided into multiple doses for purposes of administration. Consequently, single dose compositions may contain such amounts or submultiples thereof to make up the daily dose. When co-administered with other nicotinic ligands (agonist, partial agonists), the dose ranges of the compounds of this invention may be adjusted to achieve desirable efficacy and tolerability profiles.

Use with Nicotinic Acetylcholine Receptor Ligands

It has been found that the efficacy of nicotinic receptor ligands known in the art can be improved by combining the nicotinic receptor ligand, particularly an α4β2 receptor ligand (agonist, partial agonist), with compounds of the invention, i.e. a nicotinic acetylcholine receptor α4β2 subtype selective positive allosteric modulator (PAM). Such combinations are highly efficient for improving the efficacy of α4β2 ligand for treatment of pain and other disease indications such as cognitive deficits when compared to administration of an α4β2 receptor ligand alone.

Nicotinic acetylcholine ligands modulate the function by altering the activity of the receptor. Suitable compounds also can be partial agonists that partially block or partially activate the α4β2 receptor or agonists that activate the receptor. Positive allosteric modulators are compounds that potentiate receptor responses to acetylcholine without themselves triggering receptor activation or desensitization, or either, of the receptor. Nicotinic acetylcholine receptor α4β2 receptor ligands suitable for the invention can include full agonists or partial agonists, and can exhibit varying degrees of selectivity towards the α4β2 receptor.

One manner for characterizing interactions with α4β2 receptor is by assessing Kᵢ values for the displacement of [³H]-cytisine binding. Typical ligands can have Kᵢ values ranging from 1 pM to 10 μM. The [³H]-cytisine binding assays have been well reported; however, further details for carrying out the assays can be obtained in International Publication No. WO 99/32480; U.S. Pat. Nos. 5,948,793 and 5,914,328; WO 2004/018607; U.S. Pat. No. 6,809,105; WO 00/71534; and U.S. Pat. No. 6,833,370.

Accordingly, α4β2 receptor ligands suitable for the invention can be compounds of various chemical classes. Particularly, some examples of α4β2 receptor ligands suitable for the invention include, but are not limited to heterocyclic ether derivatives, for example as described in International Publication No. WO 99/32480, published Jul. 1, 1999 and further described and claimed in U.S. Pat. No. 5,948,793, issued Sep. 7, 1999, and U.S. Pat. No. 5,914,328, issued Jun. 22, 1999; N-substituted diazabicyclic derivatives, for example as described in International Publication No. WO 2004/0186107, published Sep. 23, 2004, and further described and claimed in U.S. Pat. No. 6,809,105, issued Oct. 26, 2004; heterocyclic substituted amino azacycles, for example as described in International Publication No. WO 00/71534, published Nov. 30, 2000, and further described and claimed in U.S. Pat. No. 6,833,370, issued Dec. 21, 2004; all of which are hereby incorporated by reference in their entirety. Further description and methods for preparing the compounds have been reported in patents, patent publications, and international patent publications cited.

Various forms of pain, psychiatric and neurological disorders can be treated by concurrently administering to a patient (i.e. a human) in need thereof, an α4β2 PAM and an α4β2 receptor ligand. Such combination may be especially useful in expanding the dosage range for obtaining therapeutically beneficial effects.

As used in this application, the term “concurrently administering” or “concurrent administration” refers to administering, or the administration of, respectively, an α4β2 receptor ligand to a patient, who has been prescribed (or has consumed) at least one an α4β2 PAM, at an appropriate time so that the patient's symptoms may subside. This may mean simultaneous administration of an α4β2 PAM and an α4β2 receptor ligand, or administration of the medications at different, but appropriate times. Establishing such a proper dosing schedule will be readily apparent to one skilled in the art, such as a physician treating various pain states.

The dosage range at which the α4β2 PAM and an α4β2 receptor ligand will be administered concurrently can vary widely. The specific dosage will be chosen by the patient's physician taking into account the particular compounds chosen, the severity of the patient's illness, any other medical conditions or diseases the patient is suffering from, other drugs the patient is taking and their potential to cause an interaction or adverse event, the patient's previous response to medication, and other factors.

The α4β2 PAM and an α4β2 receptor ligand should be administered concurrently in amounts that are effective to treat the patient's pain, cognitive disorder, or related condition. In more general terms, one would create a combination of the present invention by choosing a dosage of an α4β2 PAM and an α4β2 receptor ligand according to the spirit of the guidelines presented above.

The invention also is carried out by administering an α4β2 PAM together with an α4β2 receptor ligand in any manner which provides effective levels of the compounds in the body at the same time. Typically, the combination will be administered orally.

However, the invention is not limited to oral administration. The invention should be construed to cover any route of administration that is appropriate for the medications involved and for the patient. For example, transdermal administration may be very desirable for patients who are forgetful or petulant about taking oral medicine. Injections may be appropriate for patients refusing their medication. One of the drugs may be administered by one route, such as oral, and the others may be administered by the transdermal, percutaneous, intravenous, intramuscular, intranasal, or intrarectal route, in particular circumstances. The route of administration may be varied in any way, limited by the physical properties of the drugs and the convenience of the patient and the caregiver.

### Combination Use in Pain Therapy

Based on the diversity of the mechanisms underlying chronic pain (e.g. nociceptive or neuropathic, degrees of pain intensity, various etiologies etc), currently available pain medications are not efficacious in all patients or in all pain conditions. Analgesics can be broadly categorized as non-opioid analgesics (acetaminophen and non-steroidal anti-inflammatory drugs (NSAIDs)), opioid analgesics (morphine) and adjuvant analgesics or co-analgesics (antiepileptic drugs and antidepressants). In a simplified classification, non-opioid analgesics are mostly used to relieve mild to moderate nociceptive pain, adjuvant analgesics (gabapentin, pregabalin) are used to relieve neuropathic pain, and opioid analgesics are used to treat severe pain of all origins, depending on the dose prescribed.

nAChR ligands act at multiple locations throughout the pain pathway to relieve pain. Neuronal nAChRs are found on primary sensory neurons (periphery) where nociceptive information is initiated, in the cell body regions of these neurons (i.e. the dorsal root ganglion or DRG), the dorsal spinal cord where the first pain synapse is located, in the brainstem cell body regions that control descending innervation, as well as in the higher brain regions that integrate and perceive sensory information such as the thalamus and the cortex. The current theory supported by evidence from multiple sources (reviewed in Decker et al., Curr Topics Med Chem, 4: 369, 2004) is that anti-nociceptive effects of nAChR ligands are mediated by activation of brain stem nuclei with descending inhibitory inputs to the spinal cord. Additional pathways may also mediate analgesic effects of nAChR agonists in persistent or neuropathic pain.

One other aspect of the invention is the potential to enhance efficacy of other medications used for treating pain. As noted above, examples of currently used drugs include opioids, gabapentin, pregabalin, duloxetine and others. Novel mechanisms such as cannabinoids, vanilloid receptor antagonists and sodium channel blockers are also being developed for the treatment of pain. For many of these mechanisms, it is emerging that a component of efficacy may be driven by activation of descending inhibitory inputs. For example opioid analgesics can block pain transmission, in part by increasing descending inhibitory pathways to modulate pain transmission at the spinal level (Pasternack, G. W., Clin Neuropaharmcol. 16: 1, 1993; Lauretti, G. T., Expert Reviews in Neurotherapeutics, 6: 613-622. 2006). Since these drugs exert their effect via activating descending inhibitory inputs, and these pathways can be shared or commonly activated by α4β2 nAChR ligands, it is anticipated that co-administration of compounds of the invention, as α4β2 selective PAMs, can lead to enhanced efficacy of other analgesic agents by amplifying the descending inhibitory control of spinal cord activation. Thus, combining compounds of the invention with such therapeutic agents for pain affords the opportunity to create analgesic medications with either a broader or superior spectrum of efficacy that would improve the treatment of chronic pain.

### Determination of Biological Activity

One manner to characterize α4β2 positive allosteric modulator activity is by characterization in human HEK cells expressing the human nicotinic acetylcholine receptor subtype α4β2, particularly by use of Fluorescent Image Plate Reader technology. Such assay has been reported and further details for carrying out the assays can be obtained in International Publication No. WO 2006/114400. Another method to identify and characterize allosteric modulator activity is by expressing the α4β2 subunits in Xenopus oocytes or cell lines, and by measuring effects on ligand-evoked current responses as previously described in Curtis L, Buisson B, Bertrand S and Bertrand, D., 2002; Molecular Pharmacology, 61: 127-135. Other methods such as radioligand binding to assess receptor interactions may also be used.

To determine the effectiveness of representative compounds of this invention as ligands for α4β2 positive allosteric modulator activity, the compounds of the invention were evaluated according to the Calcium Flux Assay described below.

Calcium Flux Assays Using Cells Expressing nAChR Subtypes

Human embryonic kidney (HEK) 293 cells stably expressing human α4β2 or α3β4 combinations are grown to confluency in 162 cm² tissue culture flasks in DMEM media supplemented with 10% FBS and 25 μg/ml zeocin and 200 μg/ml hygromycin B. Cells expressing rat or ferret subunits may also be used. For assessing α3* or α7* selectivity, IMR-32 cells may also be used. IMR-32 neuroblastoma cells (ATCC) are grown to confluency in 162 cm² tissue culture flasks in minimum essential media supplemented with 10% FBS and 1 mM sodium pyruvate, 1% non-essential amino acids and 1% antibiotic-antimycotic. For the calcium flux assay, c cells are then dissociated using cell dissociation buffer and 100-150 μl per well of 3.5×10⁵ cells/ml cell suspension (˜50,000-100,000 cells/well) was plated into 96-well black plates (poly-D-lysine precoated) with clear bottom and maintained for 24-48 hrs in a tissue culture incubator at 37° C. under an atmosphere of 5% CO₂: 95% air. Other clonal cell lines or primary cell cultures that express endogenous α4* nicotinic receptors may also be used in this assay. Calcium flux was measured using calcium-3 assay kit (Molecular Devices, Sunnyvale, Calif.) or fluo-4 (Invitrogen). A stock solution of the dye was prepared by dissolving each vial supplied by the vendor in Hank's balanced salt solution buffer (HBSS) or 150 mM NMDG, 20 mM CaCl₂ containing 10 mM HEPES, The stock solution was diluted 1:20 using the same buffer before use. The growth media was removed from the cells. The cells were loaded with 100 μl of the dye per well and incubated at room temperature for up to one hour for HEK 293 clonal stable cell lines or 30 min-45 min at 37° C. for IMR-32 cells Fluorescence measurements were read simultaneously from all the wells by a Fluorometic Imaging Plate Reader (FLIPR) at an excitation wavelength of 480 nm and an emission wavelength of 520 nm. Baseline fluorescence was measured for the first 6 seconds at which 3× concentrations of modulator/test compounds were added to the cell plate at 50 μl and incubated for five minutes. The fluorescence intensity was captured every second for the first 1 minute followed by every 5 seconds for an additional 4 minutes. This procedure was followed by 50 μl of 4× concentration of agonist and readings were taken for a period of 3-5 minutes as described above.

The ability of test compounds to positively modulate the response (i.e., increase the response) induced by a submaximal concentration of agonist (EC_{20-30%}) such as nicotine is measured. Potentiation is measured based on peak fluorescence responses by screening compounds at fixed concentrations or in a concentration-response manner to derive EC₅₀ values. The concentration dependence of changes fluorescence responses is fitted by nonlinear regression analysis (GraphPad Prism, San Diego, Calif.) to obtain EC₅₀ values. The degree of potentiation and EC₅₀ values of the test compounds are typically calculated. To enable rank ordering of potency and efficacy, data may be normalized to a reference PAM. In general, compounds of the invention selectively potentiate α4β2 nAChRs, but not others including ganglionic receptors expressed in IMR-32 cells. At α4β2 receptors, compounds of the invention typically increase fluorescence responses to submaximal nicotine (considered as 100%) to values ranging from 120 to 500%. The EC₅₀ values of active compounds were determined by concentration response analysis (EC₅₀) range from about 10 nM to about 30 μM. The data demonstrate the compounds of the invention are α4β2 positive allosteric modulators that potentiate receptor responses to acetylcholine without themselves triggering receptor activation or desensitization, or either, of the receptor.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

What is claimed is:

1. A compound of formula (I): 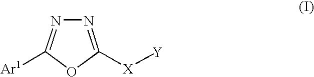
or a pharmaceutically acceptable salt or prodrug thereof, wherein
X is a bond, O, NR¹, S, or C₁-C₃ alkylene;
Y represents a monocyclic aryl, cycloalkyl, heterocycle, or heteroaryl group;
Ar¹ represents a monocyclic aryl or heteroaryl group; and
R¹ is hydrogen, alkyl, haloalkyl, or arylalkyl.

2. The compound of claim 1, wherein X is a bond.

3. The compound of claim 1, wherein X is NR¹ and R¹ is hydrogen or alkyl.

4. The compound of claim 1, wherein Y is a monocyclic aryl, monocyclic heterocycle, or a monocyclic heteroaryl group.

5. The compound of claim 1, wherein Y is phenyl, thienyl, furanyl, pyridinyl, pyrazinyl, pyrrolidinyl, or piperidinyl.

6. The compound of claim 1, wherein Ar¹ is a monocyclic heteroaryl group.

7. The compound of claim 1, wherein Ar¹ is selected from phenyl, thienyl, furanyl, pyrrolyl, pyrazolyl, thiazolyl, 1,3,4-thiadiazolyl, and pyridinyl.

8. The compound of claim 1, wherein
X is a bond;
Y is aryl, cycloalkyl, heterocycle, or heteroaryl; and
Ar¹ is monocyclic aryl or heteroaryl.

9. The compound of claim 1, wherein
X is a bond;
Y is monocyclic cycloalkyl, phenyl, thienyl, furyl, pyridinyl, pyrazinyl, pyrrolidinyl, or piperidinyl substituted with 0, 1, 2, or 3 substituents selected from alkyl, halogen, haloalkyl, hydroxy, alkoxy, haloalkoxy, nitro, and cyano; and
Ar¹ is phenyl, thienyl, furyl, pyrrolyl, pyrazolyl, thiazolyl, 1,3,4-thiadiazolyl, pyrimidinyl, pyrazinyl, or pyridinyl, substituted with 0, 1, 2, or 3 substituents selected from alkyl, alkylcarbonyl, alkylsulfonyl, alkythio, alrylalkyl, aryloxy, arylalkyloxy, halogen, haloalkyl, hydroxy, alkoxy, haloalkoxy, nitro, cyano, and NZ¹Z², wherein Z¹ and Z² are each independently hydrogen, alkyl, alkylcarbonyl, alkoxycarbonyl, aryl, arylalkyl, or formyl.

10. The compound of claim 1, wherein
X is a bond;
Y is pyridyl; and
Ar¹ is phenyl, pyrimidinyl, pyrazinyl, or pyridinyl optionally substituted with one or more of the substituents selected from the group consisting of alkyl, halogen, haloalkyl, hydroxy, alkoxy, haloalkoxy, nitro, cyano, and NZ¹Z², wherein Z¹ and Z² are each independently hydrogen, alkyl, alkylcarbonyl, alkoxycarbonyl, aryl, arylalkyl, or formyl.

11. A compound that is:
2,5-di(pyridin-3-yl)-1,3,4-oxadiazole;
2-(5-bromopyridin-3-yl)-5-(pyridin-3-yl)-1,3,4-oxadiazole;
2-(pyridin-3-yl)-5-(4-(trifluoromethyl)phenyl)-1,3,4-oxadiazole;
2-(pyridin-3-yl)-5-o-tolyl-1,3,4-oxadiazole;
2-(pyridin-3-yl)-5-m-tolyl-1,3,4-oxadiazole;
2-(pyridin-3-yl)-5-p-tolyl-1,3,4-oxadiazole;
2-(5-(pyridin-3-yl)-1,3,4-oxadiazol-2-yl)phenol;
3-(5-(pyridin-3-yl)-1,3,4-oxadiazol-2-yl)phenol;
4-(5-(pyridin-3-yl)-1,3,4-oxadiazol-2-yl)phenol;
2-(3-methoxyphenyl)-5-(pyridin-3-yl)-1,3,4-oxadiazole,
2-(4-methoxyphenyl)-5-(pyridin-3-yl)-1,3,4-oxadiazole;
2-(2-fluorophenyl)-5-(pyridin-3-yl)-1,3,4-oxadiazole;
2-(3-fluorophenyl)-5-(pyridin-3-yl)-1,3,4-oxadiazole;
2-(4-fluorophenyl)-5-(pyridin-3-yl)-1,3,4-oxadiazole;
2-(2-chlorophenyl)-5-(pyridin-3-yl)-1,3,4-oxadiazole;
2-(3-chlorophenyl)-5-(pyridin-3-yl)-1,3,4-oxadiazole;
2-(4-chlorophenyl)-5-(pyridin-3-yl)-1,3,4-oxadiazole;
2-(2-bromophenyl)-5-(pyridin-3-yl)-1,3,4-oxadiazole;
2-(3-bromophenyl)-5-(pyridin-3-yl)-1,3,4-oxadiazole;
2-(4-bromophenyl)-5-(pyridin-3-yl)-1,3,4-oxadiazole;
3-(5-(pyridin-3-yl)-1,3,4-oxadiazol-2-yl)benzonitrile;
4-(5-(pyridin-3-yl)-1,3,4-oxadiazol-2-yl)benzonitrile;
N,N-dimethyl-3-(5-(pyridin-3-yl)-1,3,4-oxadiazol-2-yl)aniline,
N,N-dimethyl-4-(5-(pyridin-3-yl)-1,3,4-oxadiazol-2-yl)aniline;
2-(pyridin-3-yl)-5-(3-(trifluoromethyl)phenyl)-1,3,4-oxadiazole;
2-(pyridin-3-yl)-5-(3-(trifluoromethoxy)phenyl)-1,3,4-oxadiazole;
2-(4-phenoxyphenyl)-5-(pyridin-3-yl)-1,3,4-oxadiazole;
2-(4-(benzyloxy)phenyl)-5-(pyridin-3-yl)-1,3,4-oxadiazole;
2-(3,4-dimethylphenyl)-5-(pyridin-3-yl)-1,3,4-oxadiazole;
2-(3,5-dimethylphenyl)-5-(pyridin-3-yl)-1,3,4-oxadiazole;
2-(2,5-dimethylphenyl)-5-(pyridin-3-yl)-1,3,4-oxadiazole;
2-(2,4-dimethylphenyl)-5-(pyridin-3-yl)-1,3,4-oxadiazole;
2-(3,4-dimethylphenyl)-5-(pyridin-3-yl)-1,3,4-oxadiazole;
2-(2,3-dimethoxyphenyl)-5-(pyridin-3-yl)-1,3,4-oxadiazole;
2-(2,4-dimethoxyphenyl)-5-(pyridin-3-yl)-1,3,4-oxadiazole;
2-(2,5-dimethoxyphenyl)-5-(pyridin-3-yl)-1,3,4-oxadiazole;
2-(2,4-dimethoxyphenyl)-5-(pyridin-3-yl)-1,3,4-oxadiazole;
2-(3,5-dimethoxyphenyl)-5-(pyridin-3-yl)-1,3,4-oxadiazole;
2-(benzo[d][1,3]dioxol-5-yl)-5-(pyridin-3-yl)-1,3,4-oxadiazole;
2-(pyridin-3-yl)-5-(3,4,5-trimethoxyphenyl)-1,3,4-oxadiazole;
2-(3,4-dichlorophenyl)-5-(pyridin-3-yl)-1,3,4-oxadiazole;
2-(2,4-dichlorophenyl)-5-(pyridin-3-yl)-1,3,4-oxadiazole;
2-(2,5-dichlorophenyl)-5-(pyridin-3-yl)-1,3,4-oxadiazole;
2-(3,4-dichlorophenyl)-5-(pyridin-3-yl)-1,3,4-oxadiazole;
5-methyl-2-(5-(pyridin-3-yl)-1,3,4-oxadiazol-2-yl)phenol;
2-methyl-5-(5-(pyridin-3-yl)-1,3,4-oxadiazol-2-yl)phenol;
2-(3-fluoro-2-methylphenyl)-5-(pyridin-3-yl)-1,3,4-oxadiazole;
2-(5-fluoro-2-methylphenyl)-5-(pyridin-3-yl)-1,3,4-oxadiazole;
2-(3-fluoro-4-methylphenyl)-5-(pyridin-3-yl)-1,3,4-oxadiazole;
2-(2,3-difluorophenyl)-5-(pyridin-3-yl)-1,3,4-oxadiazole;
2-(2,4-difluorophenyl)-5-(pyridin-3-yl)-1,3,4-oxadiazole;
2-(2,5-difluorophenyl)-5-(pyridin-3-yl)-1,3,4-oxadiazole;
2-(3,5-difluorophenyl)-5-(pyridin-3-yl)-1,3,4-oxadiazole;
1-(4-(5-(pyridin-3-yl)-1,3,4-oxadiazol-2-yl)phenyl)ethanone;
2-(4-isopropylphenyl)-5-(pyridin-3-yl)-1,3,4-oxadiazole;
2-(3-methoxy-4-methylphenyl)-5-(pyridin-3-yl)-1,3,4-oxadiazole;
2-(4-ethoxyphenyl)-5-(pyridin-3-yl)-1,3,4-oxadiazole;
2-(4-(methylthio)phenyl)-5-(pyridin-3-yl)-1,3,4-oxadiazole;
2-(3-fluoro-4-methoxyphenyl)-5-(pyridin-3-yl)-1,3,4-oxadiazole;
2-(naphthalen-1-yl)-5-(pyridin-3-yl)-1,3,4-oxadiazole;
2-(naphthalen-2-yl)-5-(pyridin-3-yl)-1,3,4-oxadiazole;
4-chloro-2-(5-(pyridin-3-yl)-1,3,4-oxadiazol-2-yl)phenol;
2-(4-tert-butylphenyl)-5-(pyridin-3-yl)-1,3,4-oxadiazole;
N-(4-(5-(pyridin-3-yl)-1,3,4-oxadiazol-2-yl)phenyl)acetamide;
2-(4-propoxyphenyl)-5-(pyridin-3-yl)-1,3,4-oxadiazole;
2-(4-isopropoxyphenyl)-5-(pyridin-3-yl)-1,3,4-oxadiazole;
2-(5-chloro-2-methoxyphenyl)-5-(pyridin-3-yl)-1,3,4-oxadiazole;
2-(4-fluoronaphthalen-1-yl)-5-(pyridin-3-yl)-1,3,4-oxadiazole;
N,N-diethyl-4-(5-(pyridin-3-yl)-1,3,4-oxadiazol-2-yl)aniline;
2-(4-butoxyphenyl)-5-(pyridin-3-yl)-1,3,4-oxadiazole;
2-(2-methoxy-4-(methylthio)phenyl)-5-(pyridin-3-yl)-1,3,4-oxadiazole;
2-(4-(methylsulfonyl)phenyl)-5-(pyridin-3-yl)-1,3,4-oxadiazole;
2-(2-chloro-5-(methylthio)phenyl)-5-(pyridin-3-yl)-1,3,4-oxadiazole;
2-(2-fluoro-5-(trifluoromethyl)phenyl)-5-(pyridin-3-yl)-1,3,4-oxadiazole;
2-(2-chloro-5-(trifluoromethyl)phenyl)-5-(pyridin-3-yl)-1,3,4-oxadiazole;
2-(2-phenethylphenyl)-5-(pyridin-3-yl)-1,3,4-oxadiazole;
2-(2-bromo-5-methoxyphenyl)-5-(pyridin-3-yl)-1,3,4-oxadiazole;
2-(5-bromo-2-chlorophenyl)-5-(pyridin-3-yl)-1,3,4-oxadiazole;
2-(2-iodophenyl)-5-(pyridin-3-yl)-1,3,4-oxadiazole;
2-(3-iodophenyl)-5-(pyridin-3-yl)-1,3,4-oxadiazole;
2-(4-iodophenyl)-5-(pyridin-3-yl)-1,3,4-oxadiazole;
2-(pyridin-3-yl)-5-(pyrimidin-5-yl)-1,3,4-oxadiazole;
2-(5-methylpyrazin-2-yl)-5-(pyridin-3-yl)-1,3,4-oxadiazole;
2-(2-chloro-6-methylpyridin-3-yl)-5-(pyridin-3-yl)-1,3,4-oxadiazole;
2-(2-methyl-6-(trifluoromethyl)pyridin-3-yl)-5-(pyridin-3-yl)-1,3,4-oxadiazole;
2-(2-(ethylthio)pyridin-3-yl)-5-(pyridin-3-yl)-1,3,4-oxadiazole;
2-(2,6-dimethoxypyridin-3-yl)-5-(pyridin-3-yl)-1,3,4-oxadiazole;
2-(2-(methylthio)pyridin-3-yl)-5-(pyridin-3-yl)-1,3,4-oxadiazole;
5-chloro-3-(5-(pyridin-3-yl)-1,3,4-oxadiazol-2-yl)pyridin-2-ol;
2-(2,6-dichloro-5-fluoropyridin-3-yl)-5-(pyridin-3-yl)-1,3,4-oxadiazole;
2-(2,5-dichloropyridin-3-yl)-5-(pyridin-3-yl)-1,3,4-oxadiazole;
2-(6-chloropyridin-3-yl)-5-(pyridin-3-yl)-1,3,4-oxadiazole;
2-(2,6-dichloropyridin-3-yl)-5-(pyridin-3-yl)-1,3,4-oxadiazole;
2-(2-chloropyridin-3-yl)-5-(pyridin-3-yl)-1,3,4-oxadiazole; and
2-(pyridin-3-yl)-5-(quinolin-3-yl)-1,3,4-oxadiazole;
or a pharmaceutically acceptable salt thereof.

12. A pharmaceutical composition comprising a therapeutically effective amount of a compound of claim 1, or a salt thereof, in a pharmaceutically acceptable carrier.

13. A method for treating or preventing a condition or disorder selected from attention deficit disorder, attention deficit hyperactivity disorder (ADHD), Alzheimer's disease (AD), bipolar disorder, mild cognitive impairment, age-associated memory impairment (AAMI), senile dementia, AIDS dementia, Pick's Disease, dementia associated with Lewy bodies, dementia associated with Down's syndrome, schizophrenia, schizoaffective disorder, smoking cessation, substance abuse, including alcohol abuse, amyotrophic lateral sclerosis, Huntington's disease, diminished CNS function associated with traumatic brain injury, infertility, lack of circulation, need for new blood vessel growth associated with wound healing, more particularly circulation around a vascular occlusion, need for new blood vessel growth associated with vascularization of skin grafts, ischemia, inflammation, sepsis, and wound healing, comprising administering a therapeutically effective amount of a compound of claim 1, or a salt thereof, to a subject in need thereof.

14. A method for treating or preventing a condition or disorder characterized by neuropsychological and cognitive dysfunction, comprising administering a therapeutically effective amount of a compound of claim 1, or a salt thereof, to a subject in need thereof.

15. A method for treating or preventing a condition or disorder selected from the acute pain, analgesic pain, post-surgical pain, chronic pain, and inflammatory pain, comprising administering a therapeutically effective amount of a compound of claim 1, or a salt thereof, to a subject in need thereof.

16. A method for treating or preventing a condition or disorder selected from neuropathic pain resulting from nerve injury following a condition selected from direct trauma to nerves, inflammation, neuritis, nerve compression, metabolic disease, infection, tumor, toxins, primary neurological diseases, or a combination thereof, comprising administering a therapeutically effective amount of a compound of claim 1, or a salt thereof, to a subject in need thereof.

17. The method of claim 16, wherein the metabolic disease is diabetes; the infection is herpes zoster or HIV; or the toxins are chemotherapy.

18. A composition, comprising:
(i) a nicotinic receptor ligand and
(ii) an α4β2 positive allosteric modulator
in admixture with at least one pharmaceutically acceptable excipient.

19. A method for use in treating or preventing pain, including neuropathic pain and cognitive disorders in a patient, comprising:
(i) administering an amount of nicotinic receptor ligand to the patient; and
(ii) administering an amount of α4β2 allosteric modulator to the patient;
wherein the amounts of (i) and (ii) together are more effective in treating pain or cognitive disorders.

20. A method for use in treating or preventing pain in a patient, comprising:
(i) administering an amount of α4β2 positive allosteric modulator ligand to the patient; and
(ii) administering a pain medication comprising a compound selected from an opioid, gabapentin, pregabalin, duloxetine, a cannabinoid ligand, a vaniolloid receptor antagonist, calcium channel blocker and a sodium channel blocker wherein a descending modulatory pathway that is shared or commonly activated via the α4β2 nicotinic receptor mechanism is activated.
